(19) United States
(12) Patent Application Publication
(10) Pub. No.: US 2004/0186170 A1
(43) Pub. Date: Sep. 23, 2004

(54) Benzofuran derivatives, pharmaceutical composition containing the same, and a process for the preparation of the active ingredient

(75) Inventors: Katalin Ivanicsne Megyeri, Budapest (HU); Aniko Miklosne Kovacs, Budapest (HU); Ildiko Nagyne Gyonos, Budapest (HU); Bela Agai, Budapest (HU); Jozsef Reiter, Budapest (HU); Gyula Simig, Budapest (HU); Endre Rivo, Budapest (HU); Zoltan Tamas Nagy, Budapest (HU); Levente Ondi, Szolnok (HU); Szabolcs Keriesz, Budapest (HU); Gabor Szenasi, Budapest (HU); Eva Schmidt, Budapest (HU); Katalin Pallagi, Budapest (HU); Istvan Gacsalyi, Budapest (HU); Istvan Gyeriyan, Budapest (HU); Tamas Szabados, Budapest (HU); Gyorgy Levay, Budakeszi (HU); Andras Egyed, Budapest (HU)

Correspondence Address: ANDERSON KILL & OLICK, P.C., 1251 Avenue of the Americas, New York, NY, 10020, US

(73) Assignee: EGIS GYOGYSZERGYAR, Budapest (HU)

(21) Appl. No.: 10/736,714

(22) Filed: Dec. 15, 2003

Related U.S. Application Data
(62) Division of application No. 09/700,345, filed on Dec. 27, 2000.
Application No. 09/700,345 is a 371 of international application No. PCT/HU99/00038, filed on May 13, 1999.

(30) Foreign Application Priority Data
May 14, 1998 (HU) P3801085
May 14, 1998 (HU) P9801086

(51) Int. Cl. A61K 31/343

(52) U.S. Cl. 514/469

(57) ABSTRACT

The present invention is a piperazinylalkylbenzofuran derivative of the formula

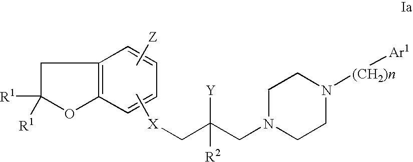

wherein

R¹ represents a C1-4 alkyl group,

R² stands for a hydrogen atom,

X means an oxygen atom,

Y is a hydroxyl group,

Z represents a hydrogen atom,

Ar′ represents a diphenylmethyl group, a pyridyl group, a partially saturated 5-membered heterocyclic group or a phenyl group,

n has a value of 0 or 1,

and pharmaceutically suitable acid addition salts thereof.

The invention refers to novel benzofuran derivatives, pharmaceutical compositions containing the same as the active ingredient, and a process for the preparation of the active ingredient. The novel compounds influence the circulatory system and the heart, furthermore have an effect on the central nervous system.

More specifically, the invention refers to a novel benzofuran derivative of the formula

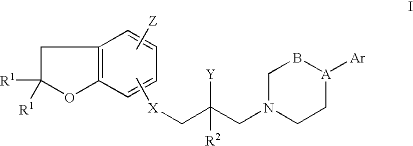

wherein

R¹ and R² represent, independently, a hydrogen atom or a C₁₋₄ alkyl group,

X stands for an oxygen atom or a sulfur atom,

Y means a hydrogen atom or a hydroxy group,

Z represents a hydrogen atom, a halo atom, a C₁₋₄ alkyl group, a C₁₋₄ alkoxy group, an amino group, a nitro group, a cyano group, a trifluoromethyl group, a group of the formula —COOR³, NHCOR³ or —SO₂NR³R⁴, wherein

R³ stands for a hydrogen atom or a C₁₋₄ alkyl group,

R⁴ is a C₁₋₄ alkyl group, or

R³ and R⁴ form, together with the adjacent nitrogen atom, a saturated or unsaturated heterocyclic group having 5 to 10 members and optionally comprising one or more nitrogen atom(s) and/or one or more oxygen atom(s) and/or one or more sulfur atom(s) as the further heteroatom(s),

A means a group of the formula CH, COH, C—CN, C—COOR³ or COR⁴, wherein R³ and R⁴ are as defined above,

B represents a methylene group, or

A forms together with B a group of the formula —C═C—,

Ar stands for a hydrogen atom, a C₁₋₄ alkyl group, a phenyl(C₁₋₄ alkyl) group, a biphenylyl group, a naphthyl group, wherein said latter species are optionally substituted by a C₁₋₄ alkoxy group or

a C₂₋₄ alkenyl group; a partially saturated, 5- or 6-membered heterocyclic group condensed with a phenyl group and containing one or two oxygen atom(s), said heterocyclic group being optionally substituted by one to three C₁₋₄ alkyl group; a 5- or

6-membered, saturated or unsaturated hetero-cyclic group containing a nitrogen atom and/or an oxygen atom and/or a sulfur atom as the heteroatom; or a phenyl group substituted by the substituents R⁵, R⁶ and R⁷, wherein

R⁵, R⁶ and R⁷ mean, independently, a hydrogen atom, a halo atom, a trifluoro-methyl group, a C₁₋₄ alkyl group, a methylenedioxy group, a phenoxy group optionally substituted by a C₁₋₄ alkoxy group or by a halo atom; a C₂₋₄ alkenyl group, a C₂₋₄ alkenyloxy group, a C₁₋₄ alkoxy group optionally substituted by a di(C₁₋₄ alkyl)amino group or by a 5- or 6-membered, saturated hetero cyclic group containing one or two nitrogen atom(s) or a nitrogen atom and an oxygen atom, wherein said heterocyclic group is optionally substituted by a C₁₋₄ alkyl group, or

A stands for a group of the formula

N—(CH₂) n—Ar′, wherein

Ar′ represents a diphenylmethyl group, a pyridyl group, a pyrimidinyl group, a naphthyl group, wherein said latter group is optionally substituted by a C₁₋₄ alkoxy group or a C₂₋₄ alkenyloxy group; a partially saturated, 5- or 6-membered heterocyclic group condensed with a phenyl group and containing one or two oxygen atom(s), said hetero-cyclic group being optionally substituted by one to three C₁₋₄ alkyl group(s); or a phenyl group substituted by the substituents R⁵, R⁶ and R⁷ wherein R⁵, R⁶ and R⁷ are as defined above,

n has a value of 0 or 1,

and pharmaceutically suitable acid addition salts thereof.

According to the literature, certain furancarboxylic amides have antidepressant properties/Yakugaku Zasshi, 97 (5), 540 (1977); C.A., 87, 152125d (1997)/, while benzo-furan derivatives having amino, amidino, thiocarboxamidino or dialkylaminoalkyl substituents on the furan ring are H₂ receptor antagonists, and, consequently, possess anantiulcer effect/publicated PCT application No. WO 86 02550; C.A., 105, 226586u (1986)/.

Tetrahydronaphthoxy derivatives having hypotensive activity are known from DE-OS No. 22 35 597. The chemical structure of the known compounds resembles to that of the piperazinylalkylbenzofuran derivatives of the formula Ia.

The aim of the invention is to prepare novel benzofuran derivatives some representants of which influencing the circulatory system and the heart function, while other representants of which having an effect on the central nervous system.

It was found that the above aim is achieved by the novel benzofuran derivatives of the formula I.

In the description and claims, in the definition of the substituents, a halo atom is, primarily, a fluoro, chloro, bromo or iodo atom, preferably a fluoro, chloro or bromo atom.

A C₁₋₄ alkyl group is a methyl, ethyl, n-propyl, isopropyl, n-butyl, sec.-butyl, tert.-butyl or isobutyl group. Preferably, a C₁₋₄ alkyl group is a methyl group.

A C₂₋₄ alkenyl group is a vinyl, allyl, methallyl or crotyl group, preferably an allyl or methallyl group.

A C₁₋₄ alkoxy group is, primarily, a methoxy, ethoxy, n-propoxy, isopropoxy or butoxy group, preferably a methoxy or isopropoxy group.

A C₂₋₄ alkenyloxy group is suitably an allyloxy or methallyloxy group.

A C₁₋₄ alkoxy group substituted by a di(C₁₋₄ alkyl)amino group is, in the first place, a 2-dimethylaminoethoxy, 3-dimethyl-aminopropoxy, 2-diethylaminoethoxy, 3-diethyl-aminopropoxy or 4-dimethylaminobutoxy group, preferably a 2-dimethylaminoethoxy group.

A partially saturated, 5- or 6-membered heterocyclic group condensed with a phenyl group and containing one or two oxygen atom(s) is a dihydrobenzofuran, benzodioxolan, dihydrobenzpyran or benzodioxan group.

A 5- or 6-membered, saturated or unsaturated heterocyclic group containing a nitrogen atom and/or an oxygen atom and/or a sulfur atom as the heteroatom is, preferably, a heterocyclic group wherein the heteroatom consists of a nitrogen atom or an oxygen atom or a sulfur atom or a nitrogen atom and an oxygen atom, and the heterocyclic ring contains no double bond or one or more double bond(s). Such a heterocyclic group is, for example, a pyrrolyl, pyrrolidinyl, piperidinyl, pyridyl, morpholinyl, furyl or thienyl group. The above heterocyclic group is suitably a thienyl group.

A 5- or 6-membered, saturated heterocyclic group containing one or two nitrogen atom(s) or a nitrogen atom and an oxygen atom is, preferably, a pyrrolidinyl, piperidinyl, piperazinyl or morpholino group, the nitrogen atom of which is linked to the carbon atom of the C₁₋₄ alkoxy group.

A saturated or unsaturated heterocyclic group having 5 to 10 members is, for example, a pyrrolidinyl, pyrrolyl, piperidinyl, pyridyl, furyl, tetrahydrofuryl, morpholinyl, piperazinyl, imidazolidinyl, pyrimidinyl, pyrazolyl, pyrazolidinyl, thienyl, hexamethyleneimine-1-yl, heptamethylene-imine-1-yl etc. group.

A pharmaceutically suitable acid addition salt is an acid addition salt formed with an inorganic acid such as hydrochloric acid, sulfuric acid, phosphoric acid etc. or with an organic acid such as acetic acid, fumaric acid, maleic acid, lactic acid, malic acid, tartaric acid etc.

The invention includes any possible isomers of the compounds of the formula I and the mixtures thereof.

A preferred subgroup of the benzofuran derivatives of the invention consists of the compounds of the formula I wherein

R¹ represents a hydrogen atom or a C₁₋₄ alkyl group,

R² stands for a hydrogen atom,

X means an oxygen atom,

Y is a hydrogen atom or a hydroxy group,

Z represents a hydrogen atom, a halo atom or a nitro group,

A stands for a group of the formula CH, COH or C—CN,

B means a methylene group, or

A forms with B a group of the formula —C═C—,

Ar represents a hydrogen atom, a benzyl group, a phenyl group substituted by substituents R⁵, R⁶ and R⁷, a biphenylyl group, a naphthyl group optionally substituted by a C₁₋₄ alkoxy group; or a thienyl group, wherein

R⁵, R⁶ and R⁷ mean, independently, a hydrogen atom, a halo atom, a trifluoro-methyl group, a C₁₋₄ alkyl group, a C₁₋₄ alkoxy group, a C₂₋₄ alkenyloxy group, a phenoxy group or a methylenedioxy group,

and pharmaceutically suitable acid addition salts thereof.

Within the above subgroup, the suitable benzofuran derivatives of the invention consist of compounds of the formula I wherein

R¹ represents a methyl group,

R² stands for a hydrogen atom,

X means an oxygen atom,

Y is a hydroxy group,

Z represents a hydrogen atom,

A is a group of the formula CH, COH or C—CN,

B stands for a methylene group, or

A forms with B a group of the formula —C═C—,

Ar represents a phenyl group optionally substituted by a halo atom, a trifluoro-methyl group, a methyl group or a methoxy group; or a methoxynaphthyl group,

and pharmaceutically suitable acid addition salts thereof.

The especially preferred benzofuran derivatives of the formula I are as follows: 1-/3-(2,2-dimethyl-2,3-dihydro-benzofuran-7-yl-oxy)-2-hydroxypropyl/-4-(3-trifluoromethyl-phenyl)-1,2,3,6-tetrahydropyridine, 1-/3-(2,2-dimethyl-2,3-dihydro-benzofuran-7-yl-oxy)-2-hydroxypropyl/-4-hydroxy-4-(3-trifluoro-methylphenyl)piperidine, 1-/3-(2,2-dimethyl-2,3-dihydro-benzofuran-7-yl-oxy)-2-hydroxypropyl/-4-hydroxy-4-(4-fluoro-phenyl)piperidine, 1-/3-(2,2-dimethyl-2,3-dihydro-benzofuran-7-yl-oxy)-2-hydroxypropyl/-4-hydroxy-4-phenyl-piperidine, 1-/3-(2,2-dimethyl-2,3-dihydro-benzofuran-7-yl-oxy)-2-hydroxypropyl/-4-hydroxy-4-(3-chloro-phenyl)piperidine, 1-/3-(2,2-dimethyl-2,3-dihydro-benzofuran-7-yl-oxy)-2-hydroxypropyl/-4-hydroxy-4-(3-methoxy-phenyl)piperidine, 1-/3-(2,2-dimethyl-2,3-dihydro-benzofuran-7-yl-oxy)-2-hydroxypropyl/-4-hydroxy-4-(4-methoxy-phenyl)piperidine, 1-/3-(2,2-dimethyl-2,3-dihydro-benzofuran-7-yl-oxy)-2-hydroxypropyl/-4-(3-trifluoromethyl-phenyl)piperidine, 1-/3-(2,2-dimethyl-2,3-dihydro-benzofuran-7-yl-oxy)-2-hydroxypropyl/-4-hydroxy-4-(4-methyl-phenyl)piperidine, 1-/3-(2,2-dimethyl-2,3-dihydro-benzofuran-7-yl-oxy)-2-hydroxypropyl/-4-cyano-4-phenyl-piperidine, 1-/3-(2,2-dimethyl-2,3-dihydro-benzofuran-7-yl-oxy)-2-hydroxypropyl/-4-hydroxy-4-(4-chloro-phenyl)piperidine or 1-/3-(2,2-dimethyl-2,3-dihydro-benzofuran-7-yl-oxy)-2-hydroxypropyl/-4-hydroxy-4-(6-methoxy-naphth-2-yl)piperidine,

and pharmaceutically suitable acid addition salts thereof.

A further preferred subgroup of the benzofuran derivatives of the invention consists of the piperazinylalkylbenzofuran derivatives of the formula

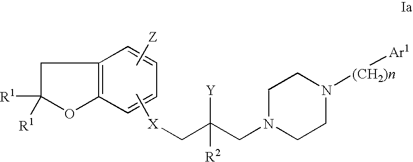

wherein

R¹ represents a C₁₋₄ alkyl group,

R² stands for a hydrogen atom,

X means an oxygen atom,

Y is a hydroxy group,

Z represents a hydrogen atom,

Ar′ represents a diphenylmethyl group, a pyridyl group, a partially saturated 5-membered heterocyclic group containing two oxygen atoms and being condensed with a phenyl group, or a phenyl group substituted by substituents R⁵, R⁶ and R⁷, wherein

R⁵, R⁶ and R⁷ mean, independently, a hydrogen atom, a halo atom, a trifluoro-methyl group, a C₁₋₄ alkyl group, a C₁₋₄ alkoxy group, or a methylenedioxy group,

n has a value of 0 or 1,

and pharmaceutically suitable acid addition salts thereof.

Within the subgroup of the formula Ia, the suitable piperazinylalkylbenzofuran derivatives of the invention consist of compounds of the formula Ia wherein

R¹ represents a methyl group,

R² stands for a hydrogen atom,

X means an oxygen atom,

Y is a hydroxy group,

z represents a hydrogen atom,

Ar′ represents a diphenylmethyl group, a pyridyl group, a benzo-1,3-dioxolanyl group or a phenyl group optionally substituted by one or two halo atom(s), one or two methyl group(s), a methylenedioxy group, a trifluoromethyl group or a methoxy group,

n has a value of 0 or 1,

and pharmaceutically suitable acid addition salts thereof.

The especially preferred benzofuran derivatives of the formula Ia are as follows: 1-/3-(2,2-dimethyl-2,3-dihydro-benzofuran-7-yl-oxy)-2-hydroxypropyl/-4-(diphenylmethyl)-piperazine, 1-/3-(−2,2-dimethyl-2,3-dihydro-benzofuran-7-yl-oxy)-2-hydroxypropyl/-4-(4-fluorophenyl)-piperazine, 1-/3-(2,2-dimethyl-2,3-dihydro-benzofuran-7-yl-oxy)-2-hydroxypropyl/-4-hydroxy-4-(3-trifluoro-methylphenyl)piperazine, 1-/3-(2,2-dimethyl-2,3-dihydro-benzofuran-7-yl-oxy)-2-hydroxypropyl/-4-(4-methoxyphenyl)-piperazine, 1-/3-(2,2-dimethyl-2,3-dihydro-benzofuran-7-yl-oxy)-2-hydroxypropyl/-4-(benzo-1,3-dioxolan-5-yl)piperazine, 1-/3-(2,2-dimethyl-2,3-dihydro-benzofuran-7-yl-oxy)-2-hydroxypropyl/-4-(4-chlorophenyl)-piperazine, 1-/3-(2,2-dimethyl-2,3-dihydro-benzofuran-7-yl-oxy)-2-hydroxypropyl/-4-benzylpiperazine, 1-/3-(2,2-dimethyl-2,3-dihydro-benzofuran-7-yl-oxy)-2-hydroxypropyl/-4-(2,4-dichlorophenyl)-piperazine, 1-/3-(2,2-dimethyl-2,3-dihydro-benzofuran-7-yl-oxy)-2-hydroxypropyl/-4-(3-chlorophenyl)-piperazine, 1-/3-(2,2-dimethyl-2,3-dihydro-benzofuran-7-yl-oxy)-2-hydroxypropyl/-4-(2-pyridyl)piperazine, 1-/3-(2,2-dimethyl-2,3-dihydro-benzofuran-7-yl-oxy)-2-hydroxypropyl/-4-(2-methoxyphenyl)-piperazine or 1-/3-(2,2-dimethyl-2,3-dihydro-benzofuran-7-yl-oxy)-2-hydroxypropyl/-4-(3-methoxyphenyl)-piperazine,

and pharmaceutically suitable acid addition salts thereof.

The compounds of the invention are prepared as follows:

a) a halide of the formula

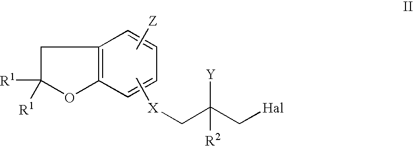

wherein R¹, R², X, Y and Z are as defined in connection with formula I, Hal represents a halo atom, is reacted with a secondary amine of the formula

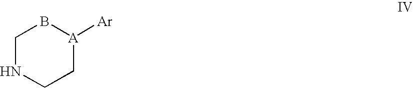

wherein A, B and Ar are as stated in connection with formula I; or

b) for the preparation of a benzofuran derivative of the formula I, wherein Y represents a hydroxy group, R¹, R², X, Z, A, B and Ar are as defined in connection with formula I, an epoxide of the formula

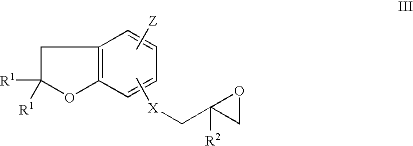

wherein R¹, R², Z and X are as defined above, is reacted with a secondary amine of the formula IV, wherein A, B and Ar are as stated above; or

c) a compound of the formula

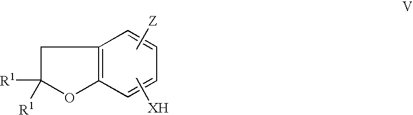

wherein R¹, X and Z are as defined in connection with formula I, is reacted with a halo compound of the formula

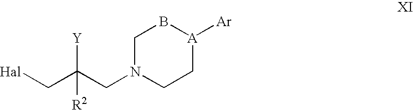

wherein R², Y, A, B and Ar are as stated in connection with formula I, Hal represents a halo atom;

d) for the preparation of a benzofuran derivative of the formula I, wherein R¹, R² X, Z, A, B and Ar are as defined in connection with formula I, a compound of the formula V, wherein R¹, X and Z are as stated above, is reacted with an epoxide of the formula

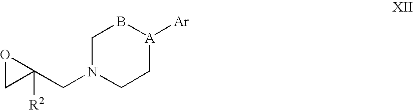

wherein R², A, B and Ar are as stated above; or

e) for the preparation of a benzofuran derivative of the formula I, wherein A forms with B a group of the formula —C═C—, R¹, R² X, Y, Z and Ar are as defined in connection with formula I, a benzofuran derivative of the formula I, wherein A stands for a group of the formula COH, B represents a methylene group, R¹, R², X, Y, Z and Ar are as stated above, is dehydrated; or

f) for the preparation of a benzofuran derivative of the formula I, wherein A represents a group of the formula COH, B stands for a methylene group, R¹, R², X, Y, Z and Ar are as defined in connection with formula I, however, Ar is other than a hydrogen atom, a ketone of the formula

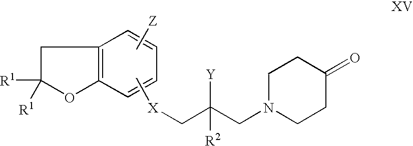

wherein R¹, R², X, Y and Z are as stated above, is reacted with an arylmagnesium halide of the formula

Hal-Mg—Ar XVI

wherein Ar is as stated above, Hal represents a halo atom, and the adduct formed is decomposed with water; or

g) for the preparation of a benzofuran derivative of the formula I, wherein A represents a group of the formula COH, B stands for a methylene group, R¹, R², X, Y, Z and Ar are as defined in connection with formula I, but Ar is other than a hydrogen atom, a ketone of the formula XV, wherein R¹, R² X, Y and Z are as stated above, is reacted with an aryl lithium compound of the formula

Li—Ar XVII

wherein Ar is as stated above, and the adduct formed is decomposed with water; or

h) for the preparation of a benzofuran derivative of the formula I, wherein A represents a group of the formula CH, B stands for a methylene group, R¹, R², X, Y, Z and Ar are as defined in connection with formula I, a compound of the formula I, wherein A forms with B a group of the formula —C═C—, R¹, R², X, Y, Z and Ar are as stated above, is hydrogenized; or

i) for the preparation of a benzofuran derivative of the formula I, wherein A represents a group of the formula CH, B stands for a methylene group, R¹, R², X, Y, Z and Ar are as defined in connection with formula I, an epoxide of the formula III, wherein R¹, R², Z and X are as stated above, is reacted with a secondary amine of the formula IV, wherein A stands for a group of the formula CHOH, B and Ar are as stated above, under dehydrating reaction conditions, and the formed compound of the formula I, wherein A forms with B a group of the formula —C═C—, R¹, R², X, Y, Z and Ar are as stated above, is hydrogenized in the reaction mixture in which it was prepared; and

if desired, an obtained base of the formula I is reacted with an inorganic or organic acid to form a pharmaceutically suitable acid addition salt thereof, or liberated from the acid addition salt with a base.

In process a) of the invention, the reaction of the halide of the formula II with the secondary amine of the formula IV is suitably performed in an excess of the corresponding secondary amine of the formula IV. However, the reaction can be carried out also in an indifferent solvent in the presence of a suitable base, eventually in a two-phase system.

In the reaction, the solvent can be for example an alcohol, preferably methanol, ethanol or isopropanol, diisopropyl ether, dioxane, acetonitrile, dimethyl formamide, dimethyl sulfoxide, halogenated solvents, preferably dichloromethane, 1,2-dichloroethane or chlorobenzene.

In the reaction, the base can be an inorganic one such as an alkali metal hydroxide or an alkali earth metal hydroxide, preferably sodium or potassium hydroxide, or an organic base, preferably a trialkylamine or a tetraalkylammonium hydroxide. An especially preferred base is triethylamine. The base is used in a 0.8-1.1 molar equivalent, preferably 0.9-1.0 molar equivalent quantity, calculated to the compound of the formula II.

The formed product of the formula I is separated from the reaction mixture by a method known in itself. Thus, if the product crystallizes from the solvent employed in the reaction mixture or can be precipitated with another solvent that is miscible with the original solvent, then the product is filtered and purified by recrystallization or chromatography.

If the product does not crystallize from the reaction mixture, then the solution is evaporated, and the residue is recrystallized from a suitable solvent.

In some cases it is convenient to subject the evaporation residue to partition between water and an organic solvent that is immiscible with water, then to make the mixture alkaline, to separate the phases, to evaporate the organic phase, and to purify the residual base by recrystallization.

In process b) of the invention, the reaction of the epoxide of the formula III with the secondary amine of the formula IV is carried out in an indifferent solvent or in an excess of the secondary amine.

The solvent for the reaction can be, for example, methanol, ethanol, isopropanol, butanol, diisopropyl ether, acetonitrile, acetone, methyl ethyl ketone, dimethyl formamide, water or mixtures thereof, preferably ethanol, isopropanol or 5-20 mass %, preferably 10 mass % solutions thereof in water.

Each mole of the epoxide of the formula III is reacted with 0.8-2.0, preferably 0.85-1.2 moles of the secondary amine of the formula Iv. If used as a base, the secondary amine of the formula IV is added directly to the reaction mixture. If a salt of the secondary amine of the formula IV is employed, the base can be liberated in the reaction mixture in situ by adding a molar equivalent quantity of inorganic or organic base calculated to the amount of the salt of the secondary amine.

As the inorganic base, primarily a 5-40 mass %, preferably 10-25 mass % solution of an alkali metal or alkali earth metal hydroxide, preferably sodium or potassium hydroxide in water is used.

As the organic base, preferably a trialkylamine or a tetraalkylammonium hydroxide, specifically triethylamine is employed.

In general, the reaction is performed at the boiling point of the solvent employed or at a lower temperature.

The product is separated from the reaction mixture as described under process a) above.

In process c) of the invention, the reaction of the compound of the formula V with the halo compound of the formula XI is carried out in an indifferent solvent in the presence of an inorganic or organic base and a phase transfer catalyst.

The inorganic base is primarily a hydroxide or carbonate of an alkali metal or alkali earth metal, preferably sodium or potassium hydroxide or potassium carbonate. The organic base is a trialkylamine or a tetraalkylammonium hydroxide, preferably triethylamine. The base is taken in 1-2, preferably 1.2 molar equivalent quantity, calculated to the compound of the formula V.

The halo compound of the formula XI is used in 1-3, preferably 1.8-2 molar equivalent quantity, calculated to the compound of the formula V.

The solvent for the reaction can be, for example, methanol, ethanol, propanol, butanol, acetone, methyl ethyl ketone, diethyl ketone, acetonitrile, dimethyl formamide, water, preferably ethanol, acetone or methyl ethyl ketone.

The phase transfer catalyst can be a tetraalkylammonium hydroxide or halide, preferably trimethylbenzylammonium hydroxide, triethylbenzylammonium hydroxide, trimethyl-benzylammonium chloride, tetrabutylammonium bromide or tetrabutylammonium hydrosulfate.

The reaction is carried out at a temperature of 40 to 100° C., preferably 60 to 80° C.

The product is separated in a manner known in itself. After the end of the reaction, the reaction mixture is, for example, evaporated to dryness under reduced pressure, the residue is subjected to partition between water and an organic solvent that is immiscible with water, the separated organic phase is dried, evaporated to dryness under reduced pressure, and the residue is purified by recrystallization from a suitable solvent or by vacuum distillation.

In process d) of the invention, the compound of the formula V is reacted with the epoxide of the formula XII in an indifferent solvent.

The solvent can be, for example, methanol, ethanol, isopropanol, butanol, diisopropyl ether, acetonitrile, acetone, methyl ethyl ketone, dimethyl formamide, water or a mixture thereof, preferably ethanol, isopropanol or a 5-20 mass %, preferably 10 mass % mixture thereof in water.

The compound of the formula V is used in 0.8-2.0, preferably 0.85-1.2 molar equivalent quantity, calculated to the epoxide of the formula XII.

In general, the reaction is carried out at the boiling point of the solvent employed, or at a lower temperature.

The product can be separated from the reaction mixture as described in connection with process c) above.

In process e) of the invention, the dehydration of the compound of the formula I, wherein A represents a group of the formula COH, B stands for a methylene group, is carried out by means of a strong mineral acid, preferably hydrochloric acid, in an indifferent solvent, preferably an alcohol, especially preferably in ethanol under heating, preferably boiling. It is convenient to use the starting compound in a solution having a concentration of 5-40%, preferably 15-25%. If the product separates from the solution after cooling, then it is filtered; in the opposite case, a solution that does not dissolve the product, however, being miscible with the solvent used in the reaction, preferably ether is added to the reaction mixture, and the crystals precipitated are filtered.

In processes f) and g) of the invention, the ketone of the formula XV is reacted with the arylmagnesium halide of the formula XVI or the aryllithium compound of the formula XVII in an indifferent aprotic organic solvent, preferably ether, tetrahydrofuran or dioxane at a temperature between −10° C. and the boiling point of the solvent, preferably 10 to 30° C. The product can be obtained from the reaction mixture—after decomposing the complex formed in the reaction with an acid and evaporating the solvent—by simple recrystallization or salt formation. If the product or the salt thereof does not crystallize, the reaction mixture decomposed with an acid is made alkaline, the product is dissolved in an organic solvent being immiscible with water, the organic phase is dried, evaporated, and the base obtained is purified by crystallization or chromatography.

In processes h) and i) of the invention, the compound of the formula I, wherein A forms with B a group of the formula —C═C—, is conveniently reduced by catalytic hydrogenation using a noble metal catalyst on a carbon carrier, preferably palladium on carbon, especially preferably 10% palladium on carbon catalyst in an alcohol, preferably methanol. After removing the catalyst by filtration, the product is separated by evaporating the solvent and crystallizing the residue. As an alternative, the evaporation residue can be converted to a salt.

If the product or the salt thereof does not crystallize, the reaction mixture is subjected to partition between water and an organic solvent being immiscible with water, the organic phase is dried, evaporated, and the residual base is purified by crystallization or chromatography.

The reduction can be performed also in the reaction mixture in which the starting compound was prepared.

The halides of the formula II are novel compounds, thus, the invention includes these compounds, too.

The halides of the formula II can be prepared by reacting a compound of the formula V with a dihaloalkane of the formula

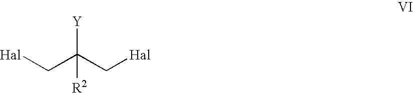

wherein Y, R² and Hal are as defined above.

Alternatively, the halide of the formula II can be prepared by reacting a compound; of the formula V with a haloalkanol derivative of the formula

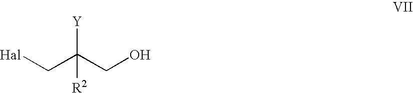

wherein Y, R² and Hal are as defined above, then converting the hydroxy group of the formed hydroxyalkyl derivative of the formula

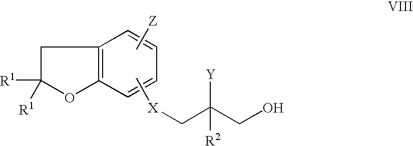

wherein R¹, R², X, Y and Z are as defined above, to a halo atom.

The compound of the formula V is reacted with the dihaloalkane of the formula VI in an indifferent solvent in the presence of an inorganic or organic base and a phase transfer catalyst.

The inorganic base is, primarily, an alkali metal or alkali earth metal hydroxide or carbonate, preferably sodium or potassium hydroxide or potassium carbonate. The organic base is a trialkylamine or a tetraalkylammonium hydroxide, preferably triethylamine. The base is employed in 1 to 1.5 molar quantity, calculated to the compound of the formula V.

The dihaloalkane of the formula VI is taken in 1-3, preferably 1.8-2 molar equivalent quantity, calculated to the compound of the formula V.

In the reaction, the solvent can be, for example, methanol, ethanol, propanol, butanol, acetone, methyl ethyl ketone, acetonitrile, dimethyl formamide, water, preferably ethanol, acetone or methyl ethyl ketone.

The phase transfer catalyst can be a tetraalkylammonium hydroxide or halide, preferably trimethylbenzylammonium hydroxide, triethylbenzylammonium hydroxide, trimethyl-benzylammonium chloride, tetrabutylammonium bromide or tetrabutylammonium hydrosulfate.

The reaction can be carried out at a temperature of 40 to 100° C., preferably 60 to 80° C.

The product is separated from the reaction mixture in a manner known in itself. For example, after the end of the reaction, the reaction mixture is evaporated to dryness under reduced pressure, the residue is subjected to partition between water and an organic solvent being immiscible with water, the organic phase is separated, dried, evaporated to dryness under reduced pressure, and the residue is purified by recrystallization from a suitable solvent or by vacuum distillation.

The reaction of the compound of the formula V with the haloalkanol derivative of the formula VII is carried out in an analogous manner as described in connection with the reaction of the compound of the formula V with the dihaloalkane of the formula VI.

The formed hydroxyalkyl derivatives of the formula VIII are converted to the desired halides of the formula II using a halogenating agent such as thionyl chloride, phosphorus oxychloride, phosphorus pentachloride, thionyl bromide, phosphorus oxybromide, phosphorus pentabromide, phosphorus pentaiodide, phosphorus tribromide, preferably thionyl chloride.

The halogenating agent is used in an excess of 1-4 moles, preferably 1.2-2.2 moles for each mole of the starting hydroxyalkyl derivative of the formula VIII.

The reaction is carried out in an indifferent solvent, preferably halogenated alkanes, especially chloroform, dichloromethane or 1,2-dichloroethane, or an excess of the halogenating agent is used as the solvent.

After evaporating the solvent, the product is separated in the same manner as described in connection with the reaction of the compound of the formula V and the dihaloalkane of the formula VI.

Some of the epoxides of the formula III are known from the literature/Japane Patent Application published under No. J6 0258-174-A; C.A., 104, 207136k (1986)/. They are prepared from the compounds of the formula V by reaction with epichlorohydrin in alkaline medium. As an alternative, they can be prepared by reacting the compound of the formula V with a halide of the formula

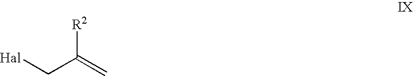

wherein R² and Hal are as defined above, and oxidizing the formed allyl derivative of the formula

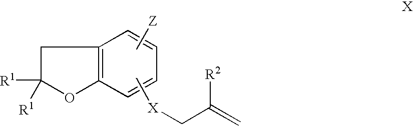

wherein R¹, R², X and Z are as stated above.

The compound of the formula V is reacted with 1-3 molar equivalent quantity of epichlorohydrin in the presence of an alkali metal or alkali earth metal hydroxide, preferably sodium or potassium hydroxide used in 1-3 molar equivalent quantity.

The reaction is carried out in methanol, ethanol, propanol, acetone, methyl ethyl ketone, acetonitrile, dimethyl formamide, water or a mixture thereof, preferably aqueous methanol or aqueous ethanol, conveniently at the boiling point of the solvent or at a lower temperature.

The epoxide of the formula III that forms in the reaction can be separated in a manner known in itself. For example, after the end of the reaction, the reaction mixture is evaporated to dryness under reduced pressure, the residue is subjected to partition between water and an organic solvent being immiscible with water, the separated organic phase is dried, and evaporated to dryness under reduced pressure. In general, the residue is pure enough for the further reactions. If necessary, the product can be purified by chromatography or crystallization.

One of the allyl derivatives of the formula X, wherein X represents an oxygen atom, R¹ stands for a methyl group, R² means a hydrogen atom and Z is a hydrogen atom, is known from the literature/Aust. J. Chem., 36 (6), 1263 (1983)/. It is prepared from the compound of the formula V which is reacted with a halide of the formula IX in an alkaline medium in the presence of a phase transfer catalyst.

In the reaction, the alkaline medium is achieved by means of an alkali metal or alkali earth metal hydroxide or carbonate, preferably sodium or potassium hydroxide or potassium carbonate. The bases listed are used in 12 molar equivalent quantity.

In the reaction, the solvent is, for example, methanol, ethanol, propanol, butanol, acetone, methyl ethyl ketone, diethyl ketone, acetonitrile, dimethyl formamide, water or a mixture thereof, preferably ethanol, acetone or methyl ethyl ketone.

As the phase transfer catalyst, a tetraalkylammonium hydroxide or halide, preferably trimethylbenzylammonium hydroxide, triethylbenzylammonium hydroxide, trimethyl-benzylammonium chloride, tetrabutylammonium bromide or tetrabutylammonium hydrosulfate can be used.

The reaction is performed at a temperature of 40 to 100° C., preferably 60 to 80° C.

The product is separated in a manner known in itself. For example, after the end of the reaction, the reaction mixture is evaporated to dryness under reduced pressure, the residue is subjected to partition between water and an organic solvent being immiscible with water, the organic phase separated is dried, evaporated to dryness under reduced pressure, and the residue is purified by recrystallization from a suitable solvent or by vacuum distillation.

The allyl derivative of the formula X is oxidized to the corresponding epoxide of the formula III with an organic oxidizing agent such as m-chloroperbenzoic acid, peracetic acid, perphthalic acid, 2,3-dichloro-5,5-dicyano-1,4-benzoquinone (DDQ), preferably m-chloroperbenzoic acid at a temperature of 0 to 40° C., preferably 20 to 30° C.

In the reaction, the solvent is dichloromethane, 1,2-dichloroethane, chloroform, 1,1,2-trichloroethylene, chloro-benzene, preferably dichloromethane or 1,2-dichloroethane.

The product is separated in a manner known in itself. For example, after the end of the reaction, water is added to the reaction mixture, the solution is made alkaline by the addition of sodium carbonate, the phases are separated, the organic phase is dried, then evaporated to dryness under reduced pressure. The residue is purified by recrystallization from a suitable solvent or by chromatography.

Some of the halo compounds of the formula XI are known/Peltz, K., Protiva, M., Coll. Czech. Chem. Commun., 32 (8), 2840 (1967), U.S. Pat. No. 4,110,536; Chem. Abstr., 90, P 121596r)/. They are prepared by reacting a corresponding secondary amine of the formula IV with a corresponding dihaloalkane of the formula VI. The other halo compounds of the formula XI are also easily prepared according to the above processes.

Some of the epoxides of the formula XII are also known. They are prepared from a corresponding compound of the formula IV that is reacted with epichlorohydrin in alkaline medium/CH—P No. 474,511; Chem. Abstr., 72, 55506m (1970)/. Certain compounds of the formula XII can be also prepared by reacting a corresponding secondary amine of the formula IV with a corresponding halide of the formula IX, and converting the double bond of the formed compound of the formula

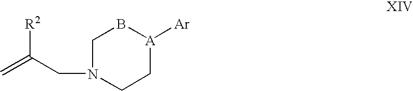

wherein R², A, B and Ar are as defined above, to an epoxy group under the conditions described in connection with the oxidation of the compounds of the formula X. The other epoxides of the formula XII can be also easily prepared according to the processes described above.

The ketones of the formula XV are novel compounds, thus, the invention includes these compounds, too. They are prepared by reacting a halide of the formula II or an epoxide of the formula III with the piperidone of the formula

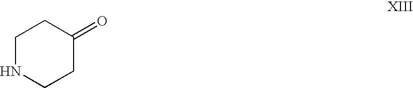

The reaction conditions are identical with those employed in processes a) and b) of the invention.

The ketone of the formula XV is reacted with the arylmagnesium halide of the formula XVI as described in the literature/J-P No. 14,632 ('67); Chem. Abstr., 68, 114618s (1968)/.

The reaction of the aryllithium derivatives of the formula XVII with the ketone of the formula XV can be also carried out in the manner known from the literature /Elpern, B., Wetteran, W., Carabates, Ph., Grunbach, L., J. Am. Chem. Soc., 80, 4916 (1958)/.

The arylmagnesium halides of the formula XVI and the aryllithium derivatives of the formula XVII are commercially available.

The secondary amines of the formula IV are, in general, commercially available, or can be easily prepared by known methods.

The biological activity of the compounds of the invention was studied in the following tests.

1. Measurement of the cardioprotective effect in ischemic rat heart (Langendorff) preparation

Cardioprotective compounds protect the myocardium from any impairment during ischemia and/or reperfusion. Of the numerous methods used for the determination of cardioprotective effect, one of the best known and often employed test consists in the isolated perfused rat heart subjected to global ischemia /Longman, S. D. and Hamilton, T. C., Medicinal Research Reviews, 12/. During global ischemia, myocardial contracture comes about due to a rise of the calcium concentration in the myocardium. The time lapsed after starting global ischemia until the beginning of contracture (i.e. time to contracture=TTC) is prolonged by several cardioprotective compounds, thus, the effectiveness of the compounds can be examined by measuring TTC.

Male Sprague-Dawley rats weighing 300 to 350 g were injected with 2500 IU (0.5 ml) of heparin i.p. and 10 minutes later the animals were anaesthetized with sodium pentobarbital/5-ethyl-5-(1-methylbutyl)-2,4,6(1H,3H,5H)-pyrimidinetrione sodium salt/ in a dose of 60 mg/kg i.p. The heart was quickly excised, and the aorta was connected to a cannula fixed to a Langendorff apparatus. The heart was perfused at a constant pressure (8000 Pa) with a modified carbogenized Krebs-Henseleit solution. The composition of the solution was the following (in mM): NaCl 118, KCl 4.7, MgSO₄ 1.6, CaCl₂ 2.5, NaHCO₃ 24.88, KH₂PO₄ 1.18, EDTA 0.5, glucose 11. Partial CO₂ pressure and pH of the solution were maintained within the physiological limits (pCO₂ 4000-4650 Pa, pH=7.3-7.45).

A hole was cut in the wall of the left atrium and a water-filled plastic balloon attached to a metal cannula was inserted into the left ventricle. End diastolic pressure of the left ventricle was set to a value between 666-1333 Pa during the equilibration period by changing the volume of the liquid in the balloon, however, later it was not modified.

After a 20 minutes' equilibration period, the hearts were perfused for 10 minutes with the vehicle (0.04% of dimethyl sulfoxide), in case of the control group, or with a solution of the test substance at 10⁻⁶ or 10⁻⁵ M concentration, in case of the treated groups. The activity of the test compound was not examined further when, at the end of the 10 minutes' period, the systolic pressure of the left ventricle was reduced by more than 20%, compared to the control value determined before the treatment.

Global ischemia was initiated by completely shutting off the perfusate flow and carbogenization for 25 minutes. The time period from the beginning of ischemia until the formation of myocardial contracture i.e. an increase of 666 Pa in left ventricular end diastolic pressure (TTC) was measured.

3 parallel experiments were performed with the test compounds at each concentration and parameters of 3 additional vehicle (0.04% dimethyl sulfoxide) treated hearts were measured along with the test substance.

Individual TTC values were averaged, and the effect of the test substances was expressed as a percentage change compared to the vehicle treated group. Compounds that prolong TTC compared to the control group are cardioprotective. As a reference substance, lemakalim i.e. (3S)-trans-3,4-dihydro-3-hydroxy-2,2-dimethyl-4-(2-oxo-1-pyrrolidinyl)-2H-1-benzpyrane-6-carbonitrile was used. The results obtained are shown in Table I.

TABLE I
TTC prolonging effect in isolated Langendorff rat hearts

| | |
|---|---|
| Compound | TTC change (in %) at |

| (No. of Example) | 10⁻⁵ M | 10⁻⁶ M |
|---|---|---|
| lemakalim | 55 | 20 |
| 9 | no data | 90 |
| 8 | 90 | 43 |
| 20 | 65 | 18 |
| 24 | no data | 47 |
| 16 | 78 | 5 |
| 26 | no data | 43 |
| 25 | 59 | 2 |
| 41 | 53 | 32 |
| 63 | 111 | −3 |
| 66 | 66 | 5 |
| 68 | 43 | 0 |
| 73 | 49 | 19 |
| 76 | 80 | 25 |

Several representants of the benzofuran derivatives examined caused a remarkably higher TTC prolongation at a concentration of 10⁻⁶ M than the reference substance lemakalim.

The compounds of the invention caused a considerable prolongation of TTC in isolated perfused rat heart during ischemia. This fact is a proof of the cardioprotective effect.

The compound of Example 9 surpasses remarkably also the effect of lemakalim determined at a concentration of 10⁻⁶ M. Thus, the cardioprotective effect of the compounds of the invention can be used for human therapy. The above cardioprotective effect raises the possibility of preventing the unfavourable effects of serious coronary arrhythmia that frequently occur in ischemic heart disease by prolonged administration of active substances. Especially the treatment of changing anginal patients seems to be suitable since in this disease considered as a state that precedes myocardial infarction, the treatment employed can reduce the rate of an irreversible myocardial damage caused by a later myocardial infarction. A further use of the compounds can be a cardioprotective therapy before surgery, for example in case of temporarily blocked coronary circulation (coronary dilatation by balloon) or stopped heart due to surgery causes. A faster and more complete regulation of heart function can be obtained by a cardioprotective treatment of the heart prepared for transplantation. In this case, the compound of the invention is added to the nutritive solution of the heart.

2. Determination of the Influence on Serotonin Neurotransmission

5-HT_{1A} Receptor Binding Assay

5-HT_{1A} receptor assay was performed according to Peroutka's method/Peroutka, S. J., J. Neurochem., 47, 529 (1986)/. The binding was determined in membrane fragments prepared from rat frontal cortex using tritium-labelled 8-hydroxy-N,N-dipropyl-2-aminotetraline as specific ligand. Non-specific binding was determined in the presence of 10 microM of 5-HT/5-hydroxytriptamine/. The final incubation volume was 250 microliters. The assay samples were incubated at 25° C. for 30 minutes. The reaction was stopped by the addition og 9 ml of an ice-cold solution of tris(hydroxymethyl)aminomethane hydrochloride having a pH value of 7.7 followed by quick filtration under reduced pressure. The filtration was carried out using a Whatman GFIB glass-fibre filter paper soaked in a 0.05% solution of polyethyleneimine for 2 to 3 hours before use. The radioactivity retained on the filters was determined by liquid scintillation counting.

The results obtained are summarized in Table II. As the reference compound, buspirone i.e. 8-{4[4-(2-pyrimidinyl)-1-piperazinyl]butyl}-8-azaspiro/4,5/decan-7,9-dione was used.

TABLE II
Effect of the compounds on 5-HT_{1A} receptor binding

| Compound | Inhibition of receptor binding |
|---|---|
| (No. of Example) | Kᵢ in nmole/litre |
| 9 | 20 |
| 20 | 6 |
| 24 | 12 |
| 30 | 5 |
| 14 | 10 |
| 16 | 3 |
| 26 | 3 |
| 23 | 12 |
| 25 | 0.7 |
| 28 | 9 |
| buspirone | 19 |

From the data of Table II it can be seen that the compounds of the invention have considerable affinity to serotonin 5-HT_{1A} receptors. Most of the compounds examined were superior to buspirone used as the reference compound.

Elevated Plus-Maze Test on Rats

The test was carried out according to a modified method of Pelow et al./J. Neurosci. Methods, 14, 149 (1985)/. The elevated plus-maze consists of two open and two 40 cm wall enclosed arms of the same size (50×15 cm) arranged in the shape of a cross. The arms of the same type are opposite to each other. The junction of the four arms forms a central square area (15×15 cm). The apparatus is made of a wooden material elevated to a height of 50 cm and illuminated by a dim light from above. Male Sprague-Dawley rats weighing 220 to 260 g were used for the experiment.

The rats were treated with the test or reference compounds 60 minutes prior to the test. The animals were then placed onto a central square area and were subjected to the test for 5 minutes. The following 4 different parameters were determined:

time spent in the open arms;

time spent in the closed arms;

number of entries into the open arms;

number of entries into the closed arms.

A compound was considered to be effective where significant increase was found either in the time spent in the open arms (in sec) or in the number of entries into the open arms when compared to the control animals (in percentage). Minimum effective doses (MED) were determined based on the time spent in the open arms for each compound examined. The results obtained are shown in Table III. Buspirone was used as the reference material.

TABLE III

| Compound | MED (in mg/kg) |
|---|---|
| (No. of Example) | p.o. |
| 16 | 1 |
| 23 | 3 |
| 24 | 3 |
| 25 | 0.3 |
| buspirone | 3 |

From Table III it can be seen that the compound of Example 25 is superior by one order of magnitude to buspirone in the above test characterizing the anxiolytic effect. It is to be noted that buspirone binds strongly to the 5-HT_{1A} receptor is widely used in the clinical practice.

On the basis of the results obtained in studies connected with the influence on the serotonin neurotransmission, the compounds of the invention can be used in various diseases primarily due to disorders of the central nervous system.

Many clinical and preclinical studies suggest that 5-HT_{1A} receptors may have a role in different pathophysiological processes. Buspirone used in our studies as a reference material and acting through 5-HT_{1A} receptors inhibited the aggressive behavior of rhesus monkeys/Tomkins, E. C., Clemento, A. J., Taylor, D. P., Perlach, J., Res. Commun. Physiol. Psychiat. Behav., 5, 337 (1980)/and indicated an anxiolytic effect in clinical trials /Goldberg, H. L. and Finnerty, R. J., Am. J. Psychiatry, 136, 1184 (1979)/. In our examinations, several compounds of the invention surpassed the anxiolytic activity of buspirone.

It is supposed that 5-HT_{1A} receptors play a role also in depression clinical patterns since it was shown that 5-HT_{1A} ligands had also an antidepressant potential in test models using animals/Porsolt, R. D., Roux, S. and Wettstein, J. G., Pharmacol. Res., 31, 169 (1995)/. A further therapeutical possibility consists in the therapy of cognitive defficiencies in case of drugs acting on the 5-HT_{1A} receptor. The administration of 8-hydroxy-N,N-dipropyl-2-aminotetraline to rats improved the memory and learning performances/Carli, M. and Samanin, R., Br. J. Pharmacol., 105, 720 (1992)/. In addition to the clinical patterns mentioned above, in case of active substances acting through the 5-HT_{1A} receptor, the use in various nutritional diseases is possible. This hypothesis is based on the fact that the 8-hydroxy-N,N-dipropyl-2-aminotetraline acting through the 5-HT_{1A} receptor, under certain conditions, enhanced the food intake, while under other conditions, it reduced the food intake/Dourish, C. T., Hutson, P. H. and Curzon, G., Psychopharmacology, 86, 197 (1985); Dourish, C. T., Hutson, P. H. and Curzon, G., Brain Res. Bull., 15, 377 (1985)/. Thus, the compounds of the invention can be effective in several clinical patterns connected with a disease of the central nervous system wherein symptoms such as anxiety, depression, cognitive defficiencies or nutritional disorder appear.

The above hypothesis is also confirmed by the following test.

3. Determination of the Anxiolytic Effect on the Basis of Vogel's Conflict Test

The anxiolytic effect was examined by the method of Vogel et al./Vogel, J. R., Beer, B., Clody, D. E., Psychopharmacologia (Berl.), 21, 1 (1971)/. Male Wistar rats weighing 180 to 200 g were left to be thirsty for 48 hours and starved for 24 hours before the test. The substances to be examined and the carriers, respectively, were administered to the animals half an hour before the examination. In the test chamber, the rats were allowed to drink from the drinking tube protruding into the chamber. Following every 20th lick, the apparatus emitted an electric shock of 0.7 mA through the drinking tube. During the test lasting for 5 minutes, the number of those electric shocks was registered which the animals accepted in order to quench their thirst. The effect of the compounds was expressed as the increase of the tolerated number of electric shocks in percentage. The minimum effective dose (MED) was determined for each compound.

Meprobamate/2-methyl-2-propyltri-methylenecarbamate/ was used as the reference substance. The results obtained are summarized in Table IV.

TABLE IV
Anxiolytic effect

| Compound | MED |
|---|---|
| (No. of Example) | in mg/kg ip. |
| 68 | 20 |
| meprobamate | 50 |

From Table IV it is apparant that the benzofurane derivative examined surpassed the effect of the meprobamate used as the reference in the Vogel's conflict test by a factor of higher than 2.

Summing up, the above tests indicate unanimously that the compounds of the invention have a valuable effect on the heart. Simultaneously, due to their mechanism of action, the compounds of the invention can be suitable for the treatment of diseases of the central nervous system such as depression, anxiety, cerebral ischemia, schizophrenia etc.

Thus, the novel benzofuran derivatives of the invention can be used as active ingredients in pharmaceutical compositions.

The pharmaceutical compositions of the invention contain a therapeutically active amount of the compound of the formula I or a pharmaceutically suitable acid addition salt thereof and one or more conventional carrier(s).

The pharmaceutical compositions of the invention are suitable for peroral, parenteral or rectal administration or for local treatment, and can be solid or liquid.

The solid pharmaceutical compositions suitable for peroral administration may be powders, capsules, tablets, film-coated tablets, microcapsules etc., and can comprise binding agents such as gelatine, sorbitol, poly(vinyl-pyrrolidone) etc.; filling agents such as lactose, glucose, starch, calcium phosphate etc.; auxiliary substances for tabletting such as magnesium stearate, talc, poly(ethyleneglycol), silica etc.; wetting agents such as sodium laurylsulfate etc. as the carrier.

The liquid pharmaceutical compositions suitable for peroral administration may be solutions, suspensions or emulsions and can comprise e.g. suspending agents such as gelatine, carboxymethylcellulose etc.; emulsifiers such as sorbitane monooleate etc.; solvents such as water, oils, glycerol, propyleneglycol, ethanol etc.; preservatives such as methyl p-hydroxybenzoate etc. as the carrier.

Pharmaceutical compositions suitable for parenteral administration consist of sterile solutions of the active ingredient, in general.

Dosage forms listed above as well as other dosage forms are known per se, see e.g. Remington's Pharmaceutical Sciences, 18th Edition, Mack Publishing Co., Easton, USA (1990).

The pharmaceutical compositions of the invention contain, in general, 0.1 to 95.0 percent by mass of a compound of the formula I or a pharmaceutically suitable acid addition salt thereof. A typical dose for adult patients amounts to 0.1 to 1000 mg of the compound of the formula I or a pharmaceutically suitable acid addition salt, daily. The above dose can be administered in one or more portions. The actual dosage depends on many factors and is determined by the doctor.

The pharmaceutical compositions of the invention are prepared by admixing a compound of the formula I or a pharmaceutically suitable acid addition salt thereof to one or more carrier(s), and converting the mixture obtained to a pharmaceutical composition in a manner known per se. Useful methods are known from the literature, e.g. Remington's Pharmaceutical Sciences.

A preferred subgroup of the pharmaceutical compositions of the invention contains a benzofuran derivative of the formula I, wherein

R¹ represents a hydrogen atom or a C₁₋₄ alkyl group,

R² stands for a hydrogen atom,

X means an oxygen atom,

Y is a hydrogen atom or a hydroxy group,

Z represents a hydrogen atom, a halo atom or a nitro group,

A stands for a group of the formula CH, COH or C—CN,

B means a methylene group, or

A forms with B a group of the formula —C═C—,

Ar represents a hydrogen atom, a benzyl group, a phenyl group substituted by substituents R⁵, R⁶ and R⁷, a biphenylyl group, a naphthyl group optionally substituted by a C₁₋₄ alkoxy group; or a thienyl group, wherein

R⁵, R⁶⁷ and R⁷ mean, independently, a hydrogen atom, a halo atom, a trifluoromethyl group, a C₁₋₄ alkyl group, a C₁₋₄ alkoxy group, a C₂₋₄ alkenyloxy group, a phenoxy group or a methylenedioxy group,

or a pharmaceutically suitable acid addition salt thereof as the active ingredient.

Suitably, within the above subgroup, the pharmaceutical compositions of the invention comprise a benzofuran derivative of the formula I, wherein

R¹ represents a methyl group,

R² stands for a hydrogen atom,

X means an oxygen atom,

Y is a hydroxy group,

Z represents a hydrogen atom,

A is a group of the formula CH, COH or C—CN,

B stands for a methylene group, or

A forms with B a group of the formula —C═C—,

Ar represents a phenyl group optionally substituted by a halo atom, a trifluoro-methyl group, a methyl group or a methoxy group; or a methoxynaphthyl group,

or a pharmaceutically suitable acid addition salt thereof as the active ingredient.

A further preferred subgroup of the pharmaceutical compositions of the invention contains a piperazinylalkylbenzofuran derivative of the formula Ia, wherein

R¹ represents a C₁₋₄ alkyl group,

R² stands for a hydrogen atom,

X means an oxygen atom,

Y is a hydroxy group,

Z represents a hydrogen atom,

Ar′ represents a diphenylmethyl group, a pyridyl group, a partially saturated 5-membered heterocyclic group containing two oxygen atoms and being condensed with a phenyl group, or a phenyl group substituted by substituents R⁵, R⁶ and R⁷, wherein

R⁵, R⁶ and R⁷ mean, independently, a hydrogen atom, a halo atom, a trifluoro-methyl group, a C₁₋₄ alkyl group, a C₁₋₄ alkoxy group, or a methylenedioxy group,

n has a value of 0 or 1,

or a pharmaceutically suitable acid addition salt thereof as the active ingredient.

Suitably, within the above subgroup, the pharmaceutical compositions of the invention contain a piperazinylalkylbenzofuran derivative of the formula Ia, wherein

R¹ represents a methyl group,

R² stands for a hydrogen atom,

X means an oxygen atom,

Y is a hydroxy group,

Z represents a hydrogen atom,

Ar′ represents a diphenylmethyl group, a pyridyl group, a benzo-1,3-dioxolanyl group or a phenyl group optionally substituted by one or two halo atom(s), one or two methyl group(s), a methylenedioxy group, a trifluoromethyl group or a methoxy group,

n has a value of 0 or 1,

or a pharmaceutically suitable acid addition salt thereof as the active ingredient.

The especially preferred pharmaceutical compositions of the invention comprise one of the following benzofuran derivatives or a pharmaceutically suitable acid addition salt thereof as the active ingredient: 1-/3-(2,2-dimethyl-2,3-dihydro-benzofuran-7-yl-oxy)-2-hydroxypropyl/-4-(3-trifluoromethyl-phenyl)-1,2,3,6-tetrahydropyridine, 1-/3-(2,2-dimethyl-2,3-dihydro-benzofuran-7-yl-oxy)-2-hydroxypropyl/-4-hydroxy-4-(3-trifluoro-methylphenyl)piperidine, 1-/3-(2,2-dimethyl-2,3-dihydro-benzofuran-7-yl-oxy)-2-hydroxypropyl/-4-hydroxy-4-(4-fluoro-phenyl)piperidine, 1-/3-(2,2-dimethyl-2,3-dihydro-benzofuran-7-yl-oxy)-2-hydroxypropyl/-4-hydroxy-4-phenyl-piperidine, 1-/3-(2,2-dimethyl-2,3-dihydro-benzofuran-7-yl-oxy)-2-hydroxypropyl/-4-hydroxy-4-(3-chloro-phenyl)piperidine, 1-/3-(2,2-dimethyl-2,3-dihydro-benzofuran-7-yl-oxy)-2-hydroxypropyl/-4-hydroxy-4-(3-methoxy-phenyl)piperidine, 1-/3-(2,2-dimethyl-2,3-dihydro-benzofuran-7-yl-oxy)-2-hydroxypropyl/-4-hydroxy-4-(4-methoxy-phenyl)piperidine, 1-/3-(2,2-dimethyl-2,3-dihydro-benzofuran-7-yl-oxy)-2-hydroxypropyl/-4-(3-trifluoromethyl-phenyl)piperidine, 1-/3-(2,2-dimethyl-2,3-dihydro-benzofuran-7-yl-oxy)-2-hydroxypropyl/-4-hydroxy-4-(4-methyl-phenyl)piperidine, 1-/3-(2,2-dimethyl-2,3-dihydro-benzofuran-7-yl-oxy)-2-hydroxypropyl/-4-cyano-4-phenyl-piperidine, 1-/3-(2,2-dimethyl-2,3-dihydro-benzofuran-7-yl-oxy)-2-hydroxypropyl/-4-hydroxy-4-(4-chloro-phenyl)piperidine, 1-/3-(2,2-dimethyl-2,3-dihydro-benzofuran-7-yl-oxy)-2-hydroxypropyl/-4-hydroxy-4-(6-methoxy-naphth-2-yl)piperidine, 1-/3-(2,2-dimethyl-2,3-dihydro-benzofuran-7-yl-oxy)-2-hydroxypropyl/-4-(diphenylmethyl)-piperazine, 1-/3-(2,2-dimethyl-2,3-dihydro-benzofuran-7-yl-oxy)-2-hydroxypropyl/-4-(4-fluorophenyl)-piperazine, 1-/3-(2,2-dimethyl-2,3-dihydro-benzofuran-7-yl-oxy)-2-hydroxypropyl/-4-hydroxy-4-(3-trifluoro-methylphenyl)piperazine, 1-/3-(2,2-dimethyl-2,3-dihydro-benzofuran-7-yl-oxy)-2-hydroxypropyl/-4-(4-methoxyphenyl)-piperazine, 1-/3-(2,2-dimethyl-2,3-dihydro-benzofuran-7-yl-oxy) -2-hydroxypropyl/-4-(benzo-1,3-dioxolan-5-yl)piperazine, 1-/3-(2,2-dimethyl-2,3-dihydro-benzofuran-7-yl-oxy)-2-hydroxypropyl/-4-(4-chlorophenyl)-piperazine, 1-/3-(2,2-dimethyl-2,3-dihydro-benzofuran-7-yl-oxy)-2-hydroxypropyl/-4-benzylpiperazine, 1-/3-(2,2-dimethyl-2,3-dihydro-benzofuran-7-yl-oxy)-2-hydroxypropyl/-4-(2,4-dichlorophenyl)-piperazine, 1-/3-(2,2-dimethyl-2,3-dihydro-benzofuran-7-yl-oxy)-2-hydroxypropyl/-4-(3-chlorophenyl)-piperazine, 1-/3-(2,2-dimethyl-2,3-dihydro-benzofuran-7-yl-oxy)-2-hydroxypropyl/-4-(2-pyridyl)piperazine, 1-/3-(2,2-dimethyl-2,3-dihydro-benzofuran-7-yl-oxy)-2-hydroxypropyl/-4-(2-methoxyphenyl)-piperazine or 1-/3-(2,2-dimethyl-2,3-dihydro-benzofuran-7-yl-oxy)-2-hydroxypropyl/-4-(3-methoxyphenyl)-piperazine.

Furthermore, the invention refers to a method for the treatment of diseases which comprises administering a therapeutically effective non-toxic amount of a benzofuran derivative of the formula I or a pharmaceutically suitable acid addition salt thereof to a patient suffering from especially a heart disease or a disease of the central nervous system.

The invention is further elucidated by means of the following Examples.

Preparation of Halides of the Formula II

1) 7-(3-Bromopropyloxy)-2,2-dimethyl-2,3-dihydrobenzofuran

To a solution of 32.8 g (0.2 moles) of 2,2-dimethyl-2,3-dihydrobenzofuran-7-ol in 600 ml of acetone, 80.8 g (0.4 moles) of 1,3-dibromopropane and 83.0 g (0.6 moles) of anhydrous potassium carbonate are added, and the reaction mixture is boiled under stirring for 24 hours. After cooling, the inorganic salts are filtered, and the filtrate is evaporated to dryness under reduced pressure. The residual product is recrystallized from methanol, filtered, then dried at room temperature.

Thus, 34.7 g (61%) of the title compound are obtained, m.p.: 54-56° C.

Preparation of Epoxides of the Formula III

2) 5-Bromo-2,2-dimethyl-7-oxiranylmethoxy-2,3-dihydrobenzofuran

a) 7-Acetoxy-2,2-dimethyl-2,3-dihydro-benzofuran

To a solution of 16.4 g (0.1 moles) of 2,2-dimethyl-2,3-dihydrobenzofuran-7-ol in 40 ml of glacial acetic acid, 12.2 g (0.12 moles) of acetic anhydride are added, the reaction mixture is boiled for 30 minutes, then evaporated to dryness under reduced pressure. The residual oily product is rubbed with 60 ml of ice-water, the white crystalline product is filtered, washed with ice-water, and dried under reduced pressure.

Thus, 20.4 g (99%) of the title compound are obtained. After recrystallization from methanol, m.p.: 49-50° C.

b) 7-Acetoxy-5-bromo-2,2-dimethyl-2,3-dihydrobenzofuran

A solution of 16.0 g (0.1 moles) of bromine in 40 ml of glacial acetic acid are added, drop by drop, to a salution of 20.6 g (0.1 moles) of 7-acetoxy-2,2-dimethyl-2,3-dihydrobenzofuran in 150 ml of chloroform under stirring and cooling at a temperature of 15-20° C. in 30 minutes. The solution obtained is stirred for 15 minutes, then evaporated to dryness under reduced pressure. The residual honeylike product is rubbed with 150 ml of icewater, the crystals precipitated are filtered, then washed with ice-water until neutrality. The thus-obtained crystals are suspended in 80 ml of methanol at 0° C., filtered again, and dried under reduced pressure.

Thus, 22.0 g (77%) of the title compound are obtained. M.p.: 76° C.

c) 5-Bromo-2,2-dimethyl-2,3-dihydro-benzofuran-7-ol

24.2 g (0.085 moles) of 7-acetoxy-5-bromo-2,2-dimethyl-2,3-dihydrobenzofuran are stirred in a mixture of 70 ml of methanol and 68 ml of 10% aqueous sodium hydroxide at 20-25° C. for 3 hours. The reaction mixture is acidified with concentrated hydrochloric acid to a pH value of 2, the methanol is distilled off under reduced pressure, and the residue is extracted 3 times using 50 ml of dichloromethane each time. The combined organic phases are washed twice using 20 ml of water each time to remove traces of the acid, dried over anhydrous sodium sulfate, filtered, and evaporated to dryness under reduced pressure. The residue is rubbed with n-hexane to obtain a crystalline substance that is filtered, and dried under reduced pressure.

Thus, 19.9 g (96.4%) of the title compound are obtained. M.p.: 70° C.

d) 5-Bromo-2,2-dimethyl-7-oxiranyl-methoxy-2,3-dihydrobenzofuran

To a solution of 19.9 g (0.082 moles) of 5-bromo-2,2-dimethyl-2,3-dihydrobenzofuran-7-ol in 60 ml of 10% aqueous sodium hydroxide, 13.65 g (0.147 moles) of epichlorohydrin are added, and the reaction mixture is stirred at 45-50° C. for 3 hours. After cooling, the separated oil is dissolved in 100 ml of dichloromethane, the aqueous phase is extracted with 30 ml of dichloro-methane, the combined organic phases are extracted twice with 20 ml of water each time, dried over anhydrous sodium sulfate, filtered, and evaporated to dryness under reduced pressure.

Thus, 23.5 g (98%) of the title compound are obtained as a honeylike matter that is used directly for the preparation of the compounds of the formula I. On standing for a long time, the product crystallizes. M.p.: 46-48° C.

3) 2,2-Dimethyl-7-oxiranylmethoxy-2,3-dihydrobenzofuran

To a solution of 15.0 g (0.09 moles) of 2,2-dimethyl2,3-dihydrobenzofuran-7-ol in 100 ml of 10% aqueous sodium hydroxide, 20.0 g (0.216 moles) of epichlorohydrin are added, and the reaction mixture is stirred at 48 to 50° C. for 2.5 hours. After cooling, the oil separated is dissolved in 100 ml of dichloromethane, the aqueous phase is extracted with 50 ml of dichloromethane, the combined organic phases are extracted twice using 20 ml of water each time, dried over anhydrous sodium sulfate, filtered, and evaporated to dryness under reduced pressure. The title compound is obtained as a thick honeylike substance that is rubbed with 60 ml of n-hexane to obtain white crystals. The crystals precipitated are filtered, and dried under reduced pressure.

Thus, 19.5 g (97%) of the title compound are obtained. M.p.: 51-52° C.

4) 2,2-Dimethyl-7-oxiranylmethoxy-2,3-dihydrobenzofuran 0.35 g of 82% m-chloroperbenzoic acid are added to a solution of 0.204 g (0.001 moles) of 2,2-dimethyl-7-(2-propenyloxy)-2,3-dihydrobenzofuran in 5 ml of dichloro-methane, and the reaction mixture is stirred for 10 hours. To the reaction mixture, 5 ml of 10% aqueous sodium hydrocarbonate solution are added, the phases are separated, the organic phase is dried over anhydrous sodium sulfate, filtered, and the solvent is removed under reduced pressure. The residual oily product is purified by column chromatography (the column is filled with silica gel and eluted with a mixture of 24 volumes of dichloromethane and 1 volume of acetone).

Thus, 0.068 g (31%) of the title compound are obtained. M.p.: 49-51° C.

5) 2,2-Dimethyl-5-nitro-7-oxiranylmethoxy-2,3-dihydrobenzofuran

a) 7-Acetoxy-2,2-dimethyl-5-nitro-2,3-dihydrobenzofuran

To a solution of 16.5 g (0.08 moles) of 7-acetoxy-2,2-dimethyl-2,3-dihydrobenzofuran in 60 ml of chloroform, 6 ml (0.06 moles) of acetic anhydride are added under stirring and cooling at 15 to 20° C. in 30 minutes. To the stirred and cooled solution obtained, 4 ml (5.53 g, 0.06 moles) of concentrated nitric acid (density: 1.42; 65%) are added, drop by drop, in 30 to 40 minutes taking care that the temperature of the mixture should be within 25 to 28° C. To the reaction mixture stirred for further 10 minutes, 100 ml of ice water are added, the phases are separated, the organic phase is washed with ice water until neutral, dried over anhydrous sodium sulfate, filtered, and the solvent is removed under reduced pressure. The crystalline product obtained is suspended in 40 ml of methanol having a temperature of 0° C., filtered, and dried under reduced pressure.

Thus, 14.8 g (74%) of the title compound are obtained. M.p.: 142-143° C.

b) 2,2-Dimethyl-5-nitro-2,3-dihydro-benzofuran-7-ol

13.3 g (0.053 moles) of 7-acetoxy-2,2-dimethyl-5-nitro-2,3-dihydrobenzofuran are stirred in a mixture of 40 ml of methanol and 42.5 ml of 10% aqueous sodium hydroxide at 20 to 25° C. for 30 minutes. The obtained solution of purpur colour is acidified to a pH value of 1 with concentrated hydrochloric acid, the methanol is distilled off under reduced pressure, and the residue is extracted with 50 ml of dichloromethane. The phases are separated, the aqueous phase is extracted twice using 25 ml of dichloromethane each time. The combined organic phases are extracted twice with 20 ml of water each time to remove the acid, then dried over anhydrous sodium sulfate, filtered, and evaporated to dryness under reduced pressure. The residue is rubbed with n-hexane to obtain a crystalline substance that is filtered and dried under reduced pressure.

Thus, 10.8 g (97.8%) of the title compound are obtained. M.p.: 96-97° C.

c) 2,2-Dimethyl-5-nitro-7-oxiranylmethoxy-2,3-dihydrobenzofuran

14.5 g (0.156 moles) of epichlorohydrin are added to a solution of 10.8 g (0.052 moles) of 2,2-dimethyl-5-nitro-2,3-dihydrobenzofuran-7-ol in 62 ml of 10% aqueous sodium hydroxide, and the reaction mixture is stirred at 48 to 52° C. for 2.5 hours. After cooling, the oil separated is dissolved in 60 ml of dichloromethane, the aqueous phase is extracted twice using 60 ml of dichloromethane each time, the combined organic phases are extracted twice with 20 ml of water each time, dried over anhydrous sodium sulfate, filtered, and evaporated to dryness under reduced pressure. The residue is rubbed with n-hexane, then filtered and dried under reduced pressure.

Thus, 13.3 g (96.7%) of the title compound are obtained. M.p.: 108° C. (after recrystallization from methanol).

6) 2,2-Dimethyl-7-(2-allyloxy)-2,2-dihydrobenzofuran

To a solution of 8.20 g (0.05 moles) of 2,2-dimethyl-2,3-dihydrobenzofuran-7-ol in 100 ml of acetone, 12.1 g (0.12 moles) of allyl bromide and 20.75 g (0.15 moles) of anhydrous potassium carbonate are added, and the reaction mixture is boiled for 12 hours under stirring. After cooling, the inorganic salts are filtered, and the filtrate is evaporated under reduced pressure to remove the solvent. The residual oily product is purified by vacuum distillation.

Thus, 9.20 g (90%) of the title compound are obtained. B.p.: 77° C./53.3 Pa.

7) 2,2-Dimethyl-7-(2-allyloxy)-2,3-dihydrobenzofuran

To a solution of 8.20 g (0.05 moles) of 2,2-dimethyl-2,3-dihydrobenzofuran-7-ol in 100 ml of acetone, 7.63 g (0.10 moles) of allyl chloride and 20.7 g (0.15 moles) of anhydrous potassium carbonate are added, and the reaction mixture is boiled for 12 hours under stirring. After cooling, the inorganic salts are filtered, and the filtrate is evaporated under reduced pressure to remove the solvent. The residual oily product is purified by vacuum distillation.

Thus, 8.90 g (87%) of the title compound are obtained. B.p.: 77° C./53.3 Pa.

Preparation of Compounds of the Formula X

8) 2,2-Dimethyl-7-(2-methyl-2-propenyloxy)-2,3-dihydrobenzofuran

To a solution of 24.6 g (0.15 moles) of 2,2-dimethyl-2,3-dihydrobenzofuran-7-ol in 350 ml of acetone, 27.1 g (0.3 moles) of methallyl chloride and 62.25 g (0.45 moles) of anhydrous potassium carbonate are added, and the reaction mixture is boiled for 22 hours under stirring. After cooling, the inorganic salts are filtered, and the filtrate is evaporated under reduced pressure to remove the solvent. The residual oily product is purified by vacuum distillation.

Thus, 25.0 g (76.5%) of the title compound are obtained. B.p.: 80-83° C./27-40 Pa.

Preparation of Benzofuran Derivatives of the Formula I

### EXAMPLE 1

1-/3-(2,2-Dimethyl-2,3-dihydrobenzofuran-7-yloxy)propyl/-4-(4-methoxyphenyl)-1,2,3,6-tetrahydropyridine hydrochloride

To a solution of 4.0 g (0.14 moles) of 7-(3-bromopropyloxy)-2,2-dimethyl-2,3-dihydro-benzofuran in 60 ml of dichloromethane, 2.28 g (0.012 moles) of 4-(4-methoxyphenyl)-1,2,3,6-tetrahydropyridine hydrochloride and 12 ml of 10% aqueous sodium hydroxide solution are added. The reaction mixture is stirred at room temperature for 12 hours, then the two phases are separated. The organic phase is extracted twice with 50 ml of water each time, dried over anhydrous sodium sulfate, filtered, then evaporated under reduced pressure to remove the solvent. The residue is dissolved in 30 ml of ethanol containing 5% of hydrogen chloride, and evaporated to dryness over a water bath under reduced pressure. The residual product is rubbed with 40 ml of ethyl acetate, and the crystalline product precipitated is filtered.

Thus, 3.75 g (72.8%) of the title compound are obtained. M.p.: 165° C.

### EXAMPLE 2

1-/3-(2,2-Dimethyl-2,3-dihydrobenzofuran-7-yloxy)propyl/-4-(4-methoxyphenyl)-1,2,3,6-tetrahydropyridine

A mixture of 2.15 g (0.005 moles) of 1-/3-(2,2-dimethyl-2,3-dihydrobenzofuran-7-yloxy)propyl/-4-(4-methoxyphenyl)-1,2,3,6-tetrahydropyridine hydrochloride, 20 ml of dichloromethane and 3 ml of 10% aqueous sodium hydroxide is stirred for 10 minutes. The two phases are separated, the organic phase is extracted twice with 5 ml of water each time, dried over anhydrous sodium sulfate, filtered, and evaporated to dryness under reduced pressure. The evaporation residue is recrystallized from a low amount of ethanol.

Thus, 1.54 g (78.5%) of the title base are obtained. M.p.: 96-97° C.

### EXAMPLE 3

J-/3-(2,2-Dimethyl-2,3-dihydrobenzofuran-7-yloxy)propyl/-4-(4-methoxyphenyl)-1,2,3,6-tetrahydropyridine hydrochloride

To a solution of 3.0 g (0.0105 moles) of 7-(3-bromopropyloxy)-2,2-dimethyl-2,3-dihydrobenzofuran in 50 ml of dichloromethane, 2.07 g (0.01 moles) of 4-hydroxy-4-(4-methoxyphenyl)piperidine and 5 ml of 10% aqueous sodium hydroxide are added, and the reaction mixture is stirred at room temperature for 14 hours. The phases are separated, the organic phase is extracted twice using 20 ml of water each time, dried over anhydrous sodium sulfate, filtered, and evaporated under reduced pressure to remove the solvent. The residue (4.15 g) is dissolved in 30 ml of ethanol containing 5% of hydrogen chloride at 40° C., the solution obtaines is maintained at the above temperature for 5 minutes, then evaporated again under reduced pressure. The residue is rubbed with 30 ml of ethyl acetate, filtered, and dried under reduced pressure.

Thus, 2.79 g (85%) of the title compound are obtained. M.p.: 165° C.

### EXAMPLE 4

1-/3-(2,2-Dimethyl-2,3-dihydrobenzofuran-7-yloxy)propyl/-4-(4-methoxyphenyl)-1,2,3,6-tetrahydropyridine hydrochloride

0.82 g (0.002 moles) of 1-/3-(2,2-dimethyl-2,3-dihydrobenzofuran-7-yloxy)-propyl/-4-(4-methoxyphenyl)-1,2,3,6-tetrahydropyridine are stirred in 10 ml of ethanol containing 5% of hydrogen chloride at 40° C. for 5 minutes, then the solution is evaporated under reduced pressure. The residue is rubbed with 30 ml of ethyl acetate, filtered, and dried under reduced pressure.

Thus, 0.75 g (87%) of the title compound are obtained. M.p.: 165° C.

### EXAMPLE 5

1-/3-(2,2-Dimethyl-2,3-dihydrobenzofuran-7-yloxy)propyl/-4-hydroxy-4-(4-methoxyphenyl)-piperidine

To a solution of 0.57 g (0.002 moles) of 7-(3-bromopropyloxy)-2,2-dimethyl-2,3-dihydrobenzofuran in 10 ml of dichloromethane, 0.41 g (0.002 moles) of 4-hydroxy-4-(4-methoxyphenyl)piperidine and 1 ml of 10% aqueous sodium hydroxide are added. The reaction mixture is stirred at room temperature for 14 hours, then the phases are separated. The organic phase is extracted twice with 5 ml of water each time, dried over anhydrous sodium sulfate, filtered, and evaporated under reduced pressure to remove the solvent. The residue is recrystallized from 3 ml of ethanol.

Thus, 0.35 g (43%) of the title compound are obtained. M.p.: 116-117° C.

### EXAMPLE 6

1-/3-(2,2-Dimethyl-2,3-dihydrobenzofuran-7-yloxy)propyl/-4-(3-trifluoromethylphenyl)-1,2,3,6-tetrahydropyridine hydrochloride

To a solution of 3.00 g (0.0105 moles) of 7-(3-bromopropyloxy)-2,2-dimethyl-2,3-dihydrobenzofuran in 50 ml of dichloromethane, 2.63 g (0.01 moles) of 4-(3-trifluoromethyl-phenyl)-1,2,3,6-tetrahydropyridine hydro-chloride and 10 ml of 10% aqueous sodium hydroxide are added. The reaction mixture is stirred at room temperature for 24 hours, then the phases are separated. The organic phase is extracted twice using 10 ml of water each time, dried over anhydrous sodium sulfate, filtered, and evaporated under reduced pressure to remove the solvent. The residue (4.68 g) is dissolved in 20 ml of ethanol containing 5% of hydrogen chloride, and evaporated to dryness again under reduced pressure. The crystalline residue is rubbed with ether, filtered, and dried under reduced pressure.

Thus, 3.86 g (82.5%) of the title compound are obtained. M.p.: 186-187° C.

### EXAMPLE 7

1-/3-(2,2-Dimethyl-2,3-dihydrobenzofuran-7-yloxy)propyl/-4-hydroxy-4-(3-trifluoromethyl-phenyl)piperidine hydrochloride

To a solution of 3.00 g (0.0105 moles) of 7-(3-bromo-propyloxy)-2,2-dimethyl-2,3-dihydrobenzofuran in 50 ml of dichloromethane, 2.45 g (0.01 moles) of 4-hydroxy-4-(3-trifluoromethylphenyl)piperidine and 5 ml of 10% aqueous sodium hydroxide are added. The reaction mixture is stirred at room temperature for 24 hours, then the phases are separated. The organic phase is extracted twice with 10 ml of water each time, dried over anhydrous sodium sulfate, filtered, and evaporated under reduced pressure to remove the solvent. The residue (4.91 g) is dissolved in 20 ml of ethanol containing 5% of hydrogen chloride, and the solution is evaporated to dryness again under reduced pressure. The crystalline residue is rubbed with ether, filtered, and dried under reduced pressure.

Thus, 3.30 g (67.9%) of the title compound are obtained. M.p.: 154-155° C.

### EXAMPLE 8

1-/3-(2,2-Dimethyl-2,3-dihydrobenzofuran-7-yloxy)-2-hydroxypropyl/-4-(3-trifluoromethyl-phenyl)-1,2,3,6-tetrahydropyridine hydrochloride

To a solution of 1.20 g (0.0055 moles) of 2,2-dimethyl-7-oxiranylmethoxy-2,3-dihydrobenzofuran in 10 ml of isopropanol, 1.30 g (0.005 moles) of 4-(3-trifluoromethyl-phenyl)-1,2,3,6-tetrahydropyridine hydro-chloride and 1.1 ml of 20% aqueous sodium hydroxide are added. The reaction mixture is boiled for 5 hours, then evaporated under reduced pressure. The residue is subjected to partition between 15 ml of dichloromethane and 10 ml of water, the organic layer is extracted twice with 10 ml of water, dried over anhydrous sodium sulfate, filtered, and evaporated under reduced pressure to remove the solvent.

The residue (2.42 g) is dissolved in 15 ml of ethanol containing 5% of hydrogen chloride, and the solution obtained is evaporated again under reduced pressure. The crystalline residue is rubbed with ether, filtered, and dried under reduced pressure.

Thus, 2.02 g (83.5%) of the title compound are obtained. M.p.: 160-162° C. (after recrystallization from isopropanol).

### EXAMPLE 9

1-/3-(2,2-Dimethyl-2,3-dihydrobenzofuran-7-yloxy)-2-hydroxypropyl/-4-hydroxy-4-(3-tri-fluoromethylphenyl)piperidine hydrochloride

To a solution of 4.40 g (0.02 moles) of 2,2-dimethyl-7-oxiranylmethoxy-2,3-dihydrobenzofuran in 30 ml of isopropanol, 4.90 g (0.02 moles) of 4-hydroxy-4-(3-tri-fluoromethylphenyl)piperidine are added. The reaction mixture is boiled for 6 hours, then evaporated to dryness under reduced pressure. The oily residue is dissolved in 15 ml of methanol, and, to the cooled solution, a mixture of 3 ml of concentrated hydrochloric acid and 3 ml of water are added at a temperature of 15 to 20° C. The crystals precipitated are filtered, washed with cold methanol, and dried under reduced pressure.

Thus, 8.06 g (86%) of the title compound are obtained. M.p.: 156-158° C. (recrystallized from isopropanol).

### EXAMPLE 10

1-/3-(2,2-Dimethyl-2,3-dihydrobenzofuran-7-yloxy)-2-hydroxypropyl/-4-(4-chlorophenyl)-1,2,3,6-tetrahydropyridine hydrochloride

To a solution of 2.80 g (0.013 moles) of 2,2-dimethyl-7-oxiranylmethoxy-2,3-dihydro-benzofuran in 20 ml of isopropanol, 2.30 g (0.01 moles) of 4-(4-chlorophenyl)-1,2,3,6-tetrahydropyridine hydrochloride and 2.2 ml of 20% aqueous sodium hydroxide are added. The reaction mixture is boiled for 6 hours, then evaporated under reduced pressure. The residue is subjected to partition between 20 ml of chloroform and 20 ml of water, the organic phase is extracted twice with 20 ml of water each time, dried over anhydrous sodium sulfate, filtered, and evaporated under reduced pressure to remove the solvent.

The residue (4.3 g) is dissolved in 15 ml of ethanol containing 10% of hydrogen chloride, and the solution obtained is evaporated to dryness again. The crystalline residue is rubbed with ether, filtered, and dried under reduced pressure.

Thus, 3.48 g (77.3%) of the title compound are obtained. M.p.: 164-166° C. (after recrystallization from isopropanol).

### EXAMPLE 11

1-/3-(2,2-Dimethyl-2,3-dihydrobenzofuran-7-yloxy)-2-hydroxypropyl/-4-(2-thienyl)-1,2,3,6-tetrahydropyridine hydrochloride

To a solution of 2.80 g (0.013 moles) of 2,2-dimethyl-7-oxiranylmethoxy-2,3-dihydro-benzofuran in 20 ml of isopropanol, 2.04 g (0.01 moles) of 4-(2-thienyl)-1,2,3,6-tetrahydropyridine hydrochloride and 2.2 ml of 20% aqueous sodium hydroxide are added. The reaction mixture is boiled for 6 hours, then evaporated under reduced pressure. The residue is subjected to partition between 20 ml of chloroform and 20 ml of water, the organic phase is extracted twice using 20 ml of water each time, dried over anhydrous sodium sulfate, filtered, and evaporated under reduced pressure to remove the solvent. The residue is dissolved in 15 ml of ethanol containing 10% of hydrogen chloride, and the solution obtained is evaporated to dryness again under reduced pressure. The crystalline residue is rubbed with ether, filtered, and dried under reduced pressure.

Thus, 3.50 g (82%) of the title compound are obtained. M.p.: 180° C.

### EXAMPLE 12

1-/3-(2,2-Dimethyl-2,3-dihydrobenzofuran-7-yloxy)-2-hydroxypropyl/-4-(4-fluorophenyl)-1,2,3,6-tetrahydropyridine hydrochloride

To a solution of 2.80 g (0.013 moles) of 2,2-dimethyl-7-oxiranylmethoxy-2,3-dihydro-benzofuran in 20 ml of isopropanol, 2.13 g (0.01 moles) of 4-(4-fluorophenyl)-1,2,3,6-tetrahydropyridine hydrochloride and 2.2 ml of 20% aqueous sodium hydroxide are added. The reaction mixture is boiled for 6 hours, then evaporated under reduced pressure. The residue is subjected to partition between 20 ml of chloroform and 20 ml of water, the organic phase is extracted twice with 20 ml of water each time, dried over anhydrous sodium sulfate, filtered, and evaporated under reduced pressure to remove the solvent. The residue is dissolved in 15 ml of ethanol containing 10% of hydrogen chloride, and the solution obtained is evaporated to dryness again under reduced pressure. The crystalline residue is rubbed with ether, filtered, and dried under reduced pressure.

Thus, 3.2 g (73.8%) of the title compound are obtained. M.p.: 153-155° C.

### EXAMPLE 13

1-/3-(2,2-Dimethyl-2,3-dihydrobenzofuran-7-yloxy)-2-hydroxypropyl/-4-benzylpiperidine hydrochloride

To a solution of 2,2-dimethyl-7-oxiranyl-methoxy-2,3-dihydrobenzofuran in 30 ml of isopropanol, 2.62 g (0.015 moles) of 4-benzyl-piperidine are added. The reaction mixture is boiled for 6 hours, then evaporated under reduced pressure. The residue is subjected to partition between 20 ml of chloroform and 20 ml of water, the organic phase is extracted twice with 20 ml of water each time, dried over anhydrous sodium sulfate, filtered, then evaporated under reduced pressure to remove the solvent. The residue is dissolved in 15 ml of ethanol containing 10% of hydrogen chloride, and the solution obtained is also evaporated to dryness under reduced pressure. The crystalline residue is rubbed with ether, filtered, and dried under reduced pressure.

Thus, 2.67 g (62%) of the title compound are obtained. M.p.: 130-132° C.

### EXAMPLE 14

1-/3-(2,2-Dimethyl-2,3-dihydrobenzofuran-7-yloxy)-2-hydroxypropyl/-4-hydroxy-4-(4-chloro-phenyl)piperidine

To a solution of 3.40 g (0.015 moles) of 2,2-dimethyl-7-oxiranylmethoxy-2,3-benzo-furan in 30 ml of isopropanol, 3.15 g (0.015 moles) of 4-hydroxy-4-(4-chlorophenyl)-piperidine are added. The reaction mixture is boiled for 6 hours, cooled, the crystals precipitated are filtered, washed with isopropanol, and dried under reduced pressure.

Thus, 5.40 g (83.4%) of the title compound are obtained. M.p.: 148-150° C. (recrystallized from acetone).

### EXAMPLE 15

1-/3-(2,2-Dimethyl-2,3-dihydrobenzofuran-7-yloxy)-2-hydroxypropyl/-4-hydroxy-4-(4-chloro-phenyl)piperidine hydrochloride

0.86 g (0.002 moles) of 1-/3-(2,2-dimethyl-2,3-dihydrobenzofuran-7-yloxy)-2-hydroxypropyl/-4-hydroxy-4-(4-chloro-phenyl)piperidine are dissolved in 10 ml of methanol, and, to the solution obtained, 2 ml of methanol containing 5% of hydrogen chloride are added under cooling with ice. The solution is evaporated to dryness under reduced pressure, the crystalline residue is rubbed with ether, filtered, then dried under reduced pressure.

Thus, 0.87 g (93%) of the title compound are obtained. M.p.: 172-174° C.

### EXAMPLE 16

1-/3-(2,2-Dimethyl-2,3-dihydrobenzofuran-7-yloxy)-2-hydroxypropyl/-4-hydroxy-4-(4-fluoro-phenyl)piperidine

To a solution of 3.4 g (0.015 moles) of 2,2-dimethyl-7-oxiranylmethoxy-2,3-dihydro-benzofuran in 30 ml of isopropanol, 2.53 g (0.013 moles) of 4-hydroxy-4-(4-fluoro-phenyl)piperidine are added, and the reaction mixture is boiled for 4 hours. The isopropanol is distilled off under reduced pressure, the residue is rubbed with 120 ml of petroleum ether (b.p.: 60° C.), the crystals precipitated are filtered, washed with a mixture of 1 volume of acetone and 4 volumes of petroleum ether, and dried under reduced pressure.

Thus, 4.55 g (84%) of the title compound are obtained. M.p.: 147-148° C. (after recrystallization from ethanol).

### EXAMPLE 17

1-/3-(2,2-Dimethyl-2,3-dihydrobenzofuran-7-yloxy)-2-hydroxypropyl/-4-hydroxy-4-(4-fluoro-phenyl)piperidine hydrochloride

0.83 g (0.002 moles) of 1-/3-(2,2-dimethyl-2,3-dihydrobenzofuran-7-yloxy)-2-hydroxypropyl/-4-hydroxy-4-(4-fluorophenyl)-piperidine are dissolved in 10 ml of methanol, and, to the solution obtained, 2 ml of methanol containing 5% of hydrogen chloride are added under cooling with ice. The solution is evaporated to dryness under reduced pressure, the crystalline residue is rubbed with ether, filtered, and dried under reduced pressure.

Thus, 0.79 g (87.5%) of the title compound are obtained. M.p.: 173-175° C.

### EXAMPLE 18

1-/3-(2,2-Dimethyl-2,3-dihydrobenzofuran-7-yloxy)-2-hydroxypropyl/-4-phenyl)-1,2,3,6-tetrahydropyridine hydrochloride

To a solution of 1.40 g (0.0063 moles) of 2,2-dimethyl-7-oxiranylmethoxy-2,3-dihydro-benzofuran in 10 ml of isopropanol, 0.98 g (0.005 moles) of 4-(4-phenyl)-1,2,3,6-tetrahydropyridine hydrochloride and 1.1 ml of 20% aqueous sodium hydroxide are added. The reaction mixture is boiled for 5 hours, then evaporated under reduced pressure. The residue is subjected to partition between 10 ml of chloroform and 10 ml of water, the organic phase is extracted twice using 10 ml of water each time, dried over anhydrous sodium sulfate, filtered, and evaporated under reduced pressure to remove the solvent. The residue is dissolved in 15 ml of ethanol containing 5% of hydrogen chloride, and the solution obtained is also evaporated under reduced pressure. The crystalline residue is rubbed with ether, filtered, and dried under reduced pressure.

Thus, 1.93 g (92.8%) of the title compound are obtained. M.p.: 166-167° C.

### EXAMPLE 19

1-/3-(2,2-Dimethyl-2,3-dihydrobenzofuran-7-yloxy)-2-hydroxypropyl/piperidine

To a solution of 2.30 g (0.0105 moles) of 2,2-dimethyl-7-oxiranylmethoxy-2,3-dihydro-benzofuran in 10 ml of isopropanol, 0.85 g (0.01 moles) of piperidine are added. The solution obtained is boiled for 3 hours, cooled, and 20 ml of isopropanol containing 5% of hydrogen chloride are added under cooling at 15 to 20° C. The reaction mixture is evaporated to dryness under reduced pressure, the residue is recrystallized from a mixture of ethanol and ether, the crystals separated are filtered, washed with ether, and dried under-reduced pressure.

Thus, 2.60 g (76%) of the title compound are obtained. M.p.: 128-130° C.

### EXAMPLE 20

1-/3-(2,2-Dimethyl-2,3-dihydrobenzofuran-7-yloxy)-2-hydroxypropyl/-4-hydroxy-4-phenyl-piperidine hydrochloride

To a solution of 3.3 g (0.015 moles) of 2,2-dimethyl-7-oxiranylmethoxy-2,3-dihydro-benzofuran in 20 ml of ethanol, 2.5 g (0.014 moles) of 4-hydroxy-4-phenylpiperidine are added. The solution obtained is boiled for 3 hours, then cooled to 15° C., and 10 ml of ethanol containing 10% of hydrogen chloride are added under cooling at 15 to 20° C. The reaction mixture is evaporated to dryness under reduced pressure, the residue is rubbed with ether, the crystals precipitated are filtered, and dried under reduced pressure.

Thus, 3.62 g (60%) of the title compound are obtained. M.p.: 176-177° C.

### EXAMPLE 21

1-/3-(2,2-Dimethyl-2,3-dihydrobenzofuran-7-yloxy)-2-hydroxypropyl/-4-hydroxy-4-phenyl-piperidine

1.4 g (0.003 moles) of 1-/3-(2,2-dimethyl-2,3-dihydrobenzofuran-7-yloxy)-2-hydroxy-propyl/-4-hydroxy-4-phenylpiperidine hydro-chloride are dissolved in 50 ml of warm water, and, to the solution obtained, 5 ml of 10% aqueous sodium hydroxide are added. The crystals precipitated are filtered, washed with water, then recrystallized from methanol.

Thus, 1.00 g (78%) of the title base are obtained. M.p.: 127-129° C.

### EXAMPLE 22

1-/3-(2,2-Dimethyl-2,3-dihydrobenzofuran-7-yloxy)-2-hydroxypropyl/-4-(2-methoxyphenyl)-piperidine hydrochloride

To a solution of 3.40 g (0.0153 moles) of 2,2-dimethyl-7-oxiranylmethoxy-2,3-dihydro-benzofuran in 30 ml of isopropanol, 3.10 g (0.015 moles) of 4-hydroxy-4-(2-methoxy-phenyl)piperidine are added. The reaction mixture is boiled for 5 hours, cooled to 15° C., and 4 ml of ethanol containing 15% of hydrogen chloride are added under cooling at 15 to 20° C. The solution obtained is evaporated to dryness under reduced pressure, and the residue is recrystallized from a mixture of ethanol and ether. The crystals precipitated are filtered, and dried under reduced pressure.

Thus, 4.73 g (68%) of the title compound are obtained. M.p.: 102-104° C.

### EXAMPLE 23

1-/3-(2,2-Dimethyl-2,3-dihydrobenzofuran-7-yloxy)-2-hydroxypropyl/-4-(6-methoxynaphth-2-yl)piperidine hydrochloride

To a solution of 2.42 g (0.011 moles) of 2,2-dimethyl-7-oxiranylmethoxy-2,3-dihydro-benzofuran in 20 ml of ethanol, 2.6 g (0.01 moles) of 4-hydroxy-4-(6-methoxynaphth-2-yl)piperidine are added. The solution obtained is boiled for 6 hours, cooled to 15° C., and 3 ml of ethanol containing 15% of hydrogen chloride are added. The reaction mixture is evaporated to dryness under reduced pressure, and the residue is rubbed with ether. The crystals precipitated are filtered, recrystallized from a mixture of ethyl acetate and ethanol, filtered again, and dried under reduced pressure.

Thus, 4.6 g (89.5%) of the title compound are obtained. M.p.: 125-127° C.

### EXAMPLE 24

1-/3-(2,2-Dimethyl-2,3-dihydrobenzofuran-7-yloxy)-2-hydroxypropyl/-4-hydroxy-4-(3-chloro-phenyl)-piperidine hydrochloride

To a solution of 2.64 g (0.012 moles) of 2,2-dimethyl-7-oxiranylmethoxy-2,3-dihydro-benzofuran in 15 ml of ethanol, 2.12 g (0.01 moles) of 4-hydroxy-4-(3-chlorophenyl)-piperidine are added. The solution obtained is boiled for 6 hours, cooled to 15° C., and 3 ml of ethanol containing 15% of hydrogen chloride are added under cooling at 15 to 20° C. The reaction mixture is evaporated to dryness under reduced pressure, and the residue is rubbed with ether. The crystals precipitated are filtered, recrystallized from a mixture of ethyl acetate and ethanol, filtered again, and dried under reduced pressure.

Thus, 3.64 g (77.8%) of the title compound are obtained. M.p.: 138-140° C.

### EXAMPLE 25

1-/3-(2,2-Dimethyl-2,3-dihydrobenzofuran-7-yloxy)-2-hydroxypropyl/-4-hydroxy-4-(3-methoxyphenyl)-piperidine hydrochloride

To a solution of 3.80 g (0.0173 moles) of 2,2-dimethyl-7-oxiranylmethoxy-2,3-dihydro-benzofuran in 30 ml of isopropanol, 3.10 g (0.015 moles) of 4-hydroxy-4-(3-methoxy-phenyl)piperidine are added. The solution obtained is boiled for 4 hours, then evaporated to dryness under reduced pressure. The residue is dissolved in 40 ml of ethyl acetate, cooled to 15° C., and 6 ml of ethanol containing 15% of hydrogen chloride are added under cooling at 15 to 20° C. The reaction mixture is evaporated to dryness under reduced pressure, and the residue is rubbed with ether. The crystals precipitated are filtered, recrystallized from a mixture of acetone and ether, filtered again, and dried under reduced pressure.

Thus, 5.90 g (84.8%) of the title compound are obtained. M.p.: 128-130° C.

### EXAMPLE 26

1-/3-(2,2-Dimethyl-2,3-dihydrobenzofuran-7-yloxy)-2-hydroxypropyl/-4-hydroxy-4-(4-methoxyphenyl)-piperidine

To a solution of 3.80 g (0.0173 moles) of 2,2-dimethyl-7-oxiranylmethoxy-2,3-dihydro-benzofuran in 20 ml of ethanol, 3.10 g (0.015 moles) of 4-hydroxy-4-(4-methoxyphenyl)-piperidine are added. The solution obtained is boiled for 4 hours, then evaporated to dryness under reduced pressure. The residue is rubbed with 40 ml of ethyl acetate, the crystals precipitated are filtered, and dried under reduced pressure.

Thus, 4.70 g (73.4%) of the title compound are obtained. M.p.: 144-146° C.

### EXAMPLE 27

1-/3-(2,2-Dimethyl-2,3-dihydrobenzofuran-7-yloxy)-2-hydroxypropyl/-4-hydroxy-4-(5-fluoro-2-methoxyphenyl)piperidine hydrochloride

To a solution of 3.80 g (0.0173 moles) of 2,2-dimethyl-7-oxiranylmethoxy-2,3-dihydro-benzofuran in 20 ml of ethanol, 3.37 g (0.015 moles) of 4-hydroxy-4-(5-fluoro-2-methoxy-phenyl)piperidine are added. The solution obtained is boiled for 6 hours, cooled to 15° C., and 4 ml of ethanol containing 15% of hydrogen chloride are added under cooling at 15 to 20° C. The reaction mixture is evaporated to dryness under reduced pressure, and the residue is rubbed with ether. The crystals precipitated are filtered, and recrystallized from 40 ml of ethyl acetate.

Thus, 5.60 g (77.5%) of the title compound are obtained. M.p.: 156-158° C.

### EXAMPLE 28

1-/3-(2,2-Dimethyl-2,3-dihydrobenzofuran-7-yloxy)-2-hydroxypropyl/-4-hydroxy-4-(3-tri-fluoromethylphenyl)piperidine hydrochloride

To a solution of 3.96 g (0.018 moles) of 2,2-dimethyl-7-oxiranylmethoxy-2,3-dihydro-benzofuran in 30 ml of ethanol, 3.34 g (0.015 moles) of 4-(3-trifluoromethylphenyl)-piperidine hydrochloride and 2 ml of 40% aqueous sodium hydroxide are added. The reaction mixture is boiled for 6 hours, then evaporated to dryness under reduced pressure. The residue is subjected to partition between 30 ml of water and 50 ml of dichloromethane, the phases are separated, the organic phase is extracted twice using 20 ml of water each time, dried over anhydrous sodium sulfate, filtered, and evaporated to dryness under reduced pressure. The residue is dissolved in 25 ml of ethanol containing 5% of hydrogen chloride, and the solution is evaporated to dryness under reduced pressure. The crystalline residue is rubbed with ether, the crystals separated are filtered, and dried under reduced pressure.

Thus, 5.15 g (77.1%) of the title compound are obtained. M.p.: 172-174° C.

### EXAMPLE 29

1-/3-(2,2-Dimethyl-2,3-dihydrobenzofuran-7-yloxy)-2-hydroxypropyl/-4-hydroxy-4-(4-methyl-phenyl)piperidine

To a solution of 3.4 g (0.0153 moles) of 2,2-dimethyl-7-oxiranylmethoxy-2,3-dihydro-benzofuran in 20 ml of ethanol, 2.86 g (0.015 moles) of 4-hydroxy-4-(4-methylphenyl)-piperidine are added. The solution obtained is boiled for 5 hours. After cooling, the solution is further cooled to 5° C. in ice bath, the crystals precipitated are filtered, and dried under reduced pressure.

Thus, 5.17 g (83.8%) of the title compound are obtained. M.p.: 138-139° C.

### EXAMPLE 30

1-/3-(2,2-Dimethyl-2,3-dihydrobenzofuran-7-yloxy)-2-hydroxypropyl/-4-hydroxy-4-(4-methyl-phenyl)piperidine hydrochloride

4.11 g (0.01 moles) of 1-/3-(2,2-dimethyl-2,3-dihydrobenzofuran-7-yloxy)-2-hydroxy-propyl/-4-hydroxy-4-(4—methylphenyl)piperidine are dissolved in 15 ml of methanol, and, to the solution cooled with ice, 12 ml of methanol containing 5% of hydrogen chloride are added. The solution is evaporated to dryness under reduced pressure, the crystalline residue is rubbed with ether, filtered, and dried under reduced pressure.

Thus, 4.17 g (93%) of the title compound are obtained. M.p.: 164-166° C.

### EXAMPLE 31

1-/3-(2,2-Dimethyl-2,3-dihydrobenzofuran-7-yloxy)-2-hydroxypropyl/-4-(4-methoxyphenyl)-1,2,3,6-tetrahydropyridine

To a solution of 3.52 g (0.016 moles) of 2,2-dimethyl-7-oxiranylmethoxy-2,3-dihydro-benzofuran in 25 ml of ethanol, 3.37 g (0.015 moles) of 4-(4-methoxyphenyl)-1,2,3,6-tetrahydropyridine hydrochloride and 2 ml of 40% aqueous sodium hydroxide are added. The reaction mixture is boiled for 6 hours, then evaporated to dryness under reduced pressure, the residue is diluted with 50 ml of water, the crystals precipitated are filtered, and dried under reduced pressure.

Thus, 6.1 g (92.8%) of the title compound are obtained. M.p.: 104-105° C. (after recrystallization from petroleum ether having a boiling point of 120° C.).

### EXAMPLE 32

1-/3-(2,2-Dimethyl-2,3-dihydrobenzofuran-7-yloxy)-2-hydroxypropyl/-4-(4-methoxyphenyl)-1,2,3,6-tetrahydropyridine hydrochloride

4.09 g (0.01 moles) of 1-/3-(2,2-dimethyl-2,3-dihydrobenzofuran-7-yloxy)-2-hydroxy-propyl/-4-(4-methoxyphenyl)-1,2,3,6-tetrahydro-pyridine are dissolved in 15 ml of methanol, and, to the solution cooled with ice, 12 ml of methanol containing 5% of hydrogen chloride are added. The solution is evaporated to dryness under reduced pressure, the crystalline residue is rubbed with ether, filtered, and dried under reduced pressure.

Thus, 4.05 g (91%) of the title compound are obtained. M.p.: 162-164° C.

### EXAMPLE 33

1-/3-(2,2-Dimethyl-2,3-dihydrobenzofuran-7-yloxy)-2-hydroxypropyl/-4-(4-methoxyphenyl)-1,2,3,6-tetrahydropyridine hydrochloride 1.10 g (0.0025 moles) of 1-/3-(2,2-dimethyl-2,3-dihydrobenzofuran-7-yloxy)-2-hydroxypropyl/-4-(4-methoxyphenyl)-1,2,3,6—-tetrahydropyridine are dissolved in 5 ml of ethanol containing 20% of hydrogen chloride, and the solution is boiled for 10 minutes. After cooling, the solution is diluted with 50 ml of ether, the crystals precipitated are filtered, washed with ether, and dried under reduced pressure.

Thus, 0.98 g (93%) of the title compound are obtained. M.p.: 161-163° C.

### EXAMPLE 34

1-/3-(2,2-Dimethyl-2,3-dihydrobenzofuran-7-yloxy)-2-hydroxypropyl/-4-hydroxy-4-(3,5-dimethyl-4-methoxyphenyl)piperidine hydrochloride

To a solution of 3.60 g (0.0165 moles) of 2,2-dimethyl-7-oxiranylmethoxy)-2,3-dihydro-benzofuran in 20 ml of ethanol, 3.45 g (0.015 moles) of 4-hydroxy-4-(3,5-dimethyl-4-methoxy-phenyl)piperidine are added. The reaction mixture is reacted at 55 to 60° C. for 2 hours, then cooled to 0°° C., and 5 ml of ethanol containing 10% of hydrogen chloride are added under cooling. The reaction mixture is evaporated to dryness under reduced pressure, and the residue is rubbed with ether. The crystals precipitated are filtered, and dried under reduced pressure.

Thus, 6.42 g (87%) of the title compound are obtained. M.p.: 92-96° C.

### EXAMPLE 35

1-/3-(2,2-Dimethyl-2,3-dihydrobenzofuran-7-yloxy)-2-hydroxypropyl/-4-hydroxy-4-(3,5-dimethyl-4-methoxyphenyl)piperidine hydrochloride

To 1.26 g (0.0057 moles) of 2,2-dimethyl-7-oxiranylmethoxy-2,3-dihydrobenzofuran, 1.18 g (0.005 moles) of 4-hydroxy-4-(3,5-dimethyl-4-methoxyphenyl)piperidine are added, and the reaction mixture is reacted at 60° C. for 1 hour. The melt is cooled, then dissolved in ether, and, to the solution obtained, 1 ml of ethanol containing 20% of hydrogen chloride are added. The crystals precipitated are filtered, washed with ether, and dried under reduced pressure.

Thus, 2.04 g (83%) of the title compound are obtained. M.p.: 94-96° C.

### EXAMPLE 36

1-/3-(2,2-Dimethyl-2,3-dihydrobenzofuran-7-yloxy)-2-hydroxypropyl/-4-hydroxy-4-(3,4-methylenedioxyphenyl)piperidine

To a solution of 2.42 g (0.011 moles) of 2,2-dimethyl-7-oxiranylmethoxy-2,3-dihydro-benzofuran in 20 ml of ethanol, 2.57 g (0.01 moles) of 4-hydroxy-4-(3,4-methylenedioxy-phenyl)piperidine hydrochloride and 4.2 ml of 10% aqueous sodium hydroxide are added. The reaction mixture is boiled for 5 hours, then evaporated to dryness under reduced pressure. The residue is subjected to partition between 20 ml of water and 50 ml of dichloro-methane, the phases are separated, the organic phase is extracted twice with 20 ml of water each time, dried over anhydrous sodium sulfate, filtered, and evaporated to dryness under reduced pressure. The residue is recrystallized from a mixture of ethyl acetate and n-hexane, the crystals precipitated are filtered, and dried under reduced pressure.

Thus, 3.01 g (73%) of the title compound are obtained. M.p.: 128-130° C.

### EXAMPLE 37

1-/3-(2,2-Dimethyl-2,3-dihydrobenzofuran-7-yloxy)-2-hydroxypropyl/-4-hydroxy-4-/4-(2-methyl-2-propenyloxy)phenyl/piperidine

To a solution of 4.8 g (0.022 moles) of 2,2-dimethyl-7-oxiranylmethoxy-2,3-dihydro-benzofuran in 20 ml of diisopropyl ether, 4.94 g (0.02 moles) of 4-hydroxy-4-/4-(2-methyl-2-propenyloxy)phenylpiperidine are added. The reaction mixture is boiled for 6 hours, then cooled to 0° C., the crystals precipitated are filtered, washed with diisopropyl ether, then dried under reduced pressure.

Thus, 7.95 g (85%) of the title compound are obtained. M.p.: 108-110° C.

### EXAMPLE 38

1-/3-(2,2-Dimethyl-2,3-dihydrobenzofuran-7-yloxy)-2-hydroxypropyl/-4-hydroxy-4-(4-biphenylyl)piperidine hydrochloride

To a solution of 2.64 g (0.012 moles) of 2,2-dimethyl-7-oxiranylmethoxy-2,3-dihydro-benzofuran in 16 ml of ethanol, 2.53 g (0.01 moles) of 4-hydroxy-4-(4-biphenylyl)piperidine are added. The reaction mixture is stirred at 70° C. for 1 hour, then evaporated to dryness under reduced pressure. The residue is dissolved in 50 ml of ether, and, to the solution obtained, 1.5 ml of ethanol containing 20% of hydrogen chloride are added under cooling. The crystals precipitated are filtered, washed with ether, then dried under reduced pressure.

Thus, 4.45 g (87%) of the title compound are obtained. M.p.: 166-167° C.

### EXAMPLE 39

1-/3-(2,2-Dimethyl-2,3-dihydrobenzofuran-7-—yloxy)-2-hydroxypropyl/-4-hydroxy-4-(4-phenoxyphenyl)piperidine

To a solution of 2.64 g (0.012 moles) of 2,2-dimethyl-7-oxiranylmethoxy-2,3-dihydro-benzofuran in 16 ml of ethanol, 2.70 g (0.01 moles) of 4-hydroxy-4-(4-phenoxyphenyl)-piperidine are added. The reaction mixture is stirred at 70° C. for 1 hour, then evaporated to dryness under reduced pressure. The residue is recrystallized from a low amount of methanol, filtered, and dried under reduced pressure.

Thus, 4.47 g (91%) of the title compound are obtained. M.p.: 113-115° C.

### EXAMPLE 40

1-/3-(2,2-Dimethyl-2,3-dihydrobenzofuran-7-yloxy)-2-hydroxypropyl/-4-hydroxy-4-(4-chloro-3-trifluoromethylphenyl)piperidine hydrochloride

To a solution of 3.90 g (0.0177 moles) of 2,2-dimethyl-7-oxiranylmethoxy-2,3-dihydrobenzofuran in 20 ml of ethanol, 4.19 g (0.015 moles) of 4-hydroxy-4-(4-chloro-3-trifluoromethylphenyl)piperidine are added. The reaction mixture is stirred at 60° C. for 2 hours, then evaporated to dryness under reduced pressure. The residue is dissolved in 60 ml of ether, and, to the solution obtained, 2.5 ml of ethanol containing 20% of hydrogen chloride are added. The crystals precipitated are filtered, washed with ether, and dried under reduced pressure.

Thus, 7.00 g (88%) of the title compound are obtained. M.p.: 146-148° C.

### EXAMPLE 41

1-/3-(2,2-Dimethyl-2,3-dihydrobenzofuran-7-yloxy)-2-hydroxypropyl/-4-cyano-4-phenyl-piperidine hydrochloride

To a solution of 0.20 g (0.005 moles) of sodium hydroxide in 15 ml of isopropanol, 1.21 g (0.0055 moles) of 2,2-dimethyl-7-oxiranylmethoxy-2,3-dihydrobenzofuran and 1.11 g (0.005 moles) of 4-cyano-4-phenyl-piperidine hydrochloride are added. The reaction mixture is boiled under stirring for 6 hours, then evaporated to dryness under reduced pressure. The residue is subjected to partition between 50 ml of water and 50 ml of dichloromethane, the phases are separated, the organic phase is extracted twice using 20 ml of water each time, dried over anhydrous sodium sulfate, filtered, and evaporated to dryness under reduced pressure. The residue is dissolved in 50 ml of ether, and, to the solution obtained, 1 ml of ethanol containing 20% of hydrogen chloride are added under cooling. The crystals precipitated are filtered, washed with ether, then dried under reduced pressure.

Thus, 1.60 g (74%) of the title compound are obtained. M.p.: 204-206° C.

### EXAMPLE 42

1-/3-(2,2-Dimethyl-2,3-dihydrobenzofuran-7-yloxy)-2-hydroxypropyl/-4-cyano-4-(3-tri-fluoromethylphenyl)piperidine hydrochloride

To a solution of 0.20 g (0.005 moles) of sodium hydroxide in 15 ml of isopropanol, 1.21 g (0.0055 moles) of 2,2-dimethyl-7-oxiranylmethoxy-2,3-dihydrobenzofuran and 1.40 g (0.005 moles) of 4-cyano-4-(3-trifluoro-methylphenyl)piperidine hydrochloride are added. The reaction mixture is boiled for 6 hours under stirring, then evaporated to dryness under reduced pressure. The residue is subjected to partition between 50 ml of water and 50 ml of dichloromethane, the phases are separated, the organic phase is extracted twice using 20 ml of water each time, dried over anhydrous sodium sulfate, filtered, and evaporated to dryness under reduced pressure. The residue is dissolved in 50 ml of ether, and, to the solution obtained, 1 ml of ethanol containing 20% of hydrogen chloride is added. The crystals precipitated is filtered, washed with ether, and dried under reduced pressure.

Thus, 1.48 g (59.2%) of the title compound are obtained. M.p.: 213-216° C.

### EXAMPLE 43

1-/3-(5-Bromo-2,2-dimethyl-2,3-dihydrobenzo-furan-7-yloxy)-2—hydroxypropyl/-4-hydroxy-4-(4-methoxyphenyl)piperidine

To a solution of 2.63 g (0.0088 moles) of 5-bromo-2,2-dimethyl-7-oxiranylmethoxy-2,3-dihydrobenzofuran in 10 ml of isopropanol, 1.66 g (0.008 moles) of 4-hydroxy-4-(4-methoxyphenyl)piperidine are added. The reaction mixture is boiled under stirring for 10 hours, then cooled to 0° C. The crystals precipitated are filtered, washed with cold isopropanol, and dried under reduced pressure.

Thus, 2.67 g (66%) of the title compound are obtained. M.p.: 120-121° C. (after recrystallization from isopropanol).

### EXAMPLE 44

1-/3-(5-Bromo-2,2-dimethyl-2,3-dihydrobenzo-furan-7-yloxy)-2—hydroxypropyl/-4-hydroxy-4-(4-chloro-3-trifluoromethylphenyl)piperidine hydrochloride

To a solution of 3.30 g (0.011 moles) of 5-bromo-2,2-dimethyl-7-oxiranylmethoxy-2,3-dihydrobenzofuran in 20 ml of ethanol, 2.80 g (0.01 moles) of 4-hydroxy-4-(4-chloro-3-trifluoromethylphenyl)piperidine are added. The reaction mixture is stirred at 60° C. for 2 hours, then evaporated to dryness under reduced pressure. The residue is dissolved in 60 ml of ether, and, to the solution obtained, 2.5 ml of ethanol containing 20% of hydrogen chloride are added under cooling. The crystals separated are filtered, washed with ether, and dried under reduced pressure.

Thus, 4.86 g (79%) of the title compound are obtained. M.p.: 108-110° C.

### EXAMPLE 45

1-/3-(2,2-Dimethyl-5-nitro-2,3-dihydrobenzo-furan-7-yloxy)-2-hydroxypropyl/-4-hydroxy-4-(4-methoxyphenyl)piperidine

To a solution of 2.62 g (0.01 moles) of 2,2-dimethyl-5-nitro-7-oxiranylmethoxy-2,3-dihydrobenzofuran in 8 ml of isopropanol, 1.86 g (0.009 moles) of 4-hydroxy-4-(4-methoxyphenyl)piperidine are added. The reaction mixture is boiled for 2 hours under stirring, then cooled to 2° C. The crystals precipitated are filtered, washed with cold isopropanol, then dried under reduced pressure.

Thus, 3.48 g (81.8%) of the title compound are obtained. M.p.: 136-138° C.

### EXAMPLE 46

1-/3-(2,2-Dimethyl-5-nitro-2,3-dihydrobenzo-furan-7-yloxy)-2—hydroxypropyl/-4-(3-trifluoromethylphenyl)-1,2,3,6-tetrahydro-pyridine hydrochloride

To a solution of 2.46 g (0.012 moles) of 2,2-dimethyl-5-nitro-7-oxiranylmethoxy-2,3-dihydrobenzofuran in 20 ml of isopropanol, 2.11 g (0.01 moles) of 4-(3-trifluoromethyl-phenyl)-1,2,3,6-tetrahydropyridine hydro-chloride and 2.2 ml of 20% aqueous sodium hydroxide are added. The reaction mixture is boiled for 5 hours under stirring, then evaporated under reduced pressure. The residue is subjected to partition between 30 ml of dichloromethane and 10 ml of water, the organic phase is extracted twice with 20 ml of water each time, dried over anhydrous sodium sulfate, filtered, and evaporated under reduced pressure to remove the solvent. The residue is dissolved in 50 ml of ethanol containing 5% os hydrogen chloride, and the solution obtained is also evaporated to dryness under reduced pressure. The crystalline residue is rubbed with ether, filtered, and dried under reduced pressure.

Thus, 3.89 g (73.7%) of the title compound are obtained. M.p.: 162-165° C.

### EXAMPLE 47

1-/3-(2,2-Dimethyl-2,3-dihydrobenzofuran-7-yloxy)-2-hydroxypropyl/-4-(6-methoxynaphth-2-yl)-1,2,3,6-tetrahydropyridine hydrochloride

A solution of 3.34 g (0.0065 moles) of 1-/3-(2,2-dimethyl-2,3-dihydrobenzofuran-7-yloxy)-2-hydroxypropyl/-4—(6-methoxynaphth-2-yl)piperidine hydrochloride in 15 ml of ethanol containing 20% of hydrogen chloride is boiled for 20 minutes, then evaporated to dryness under reduced pressure. The crystalline residue is rubbed with 30 ml of ether, filtered, and dried under reduced pressure.

Thus, 2.96 g (92%) of the title compound are obtained. M.p.: 186-188° C.

### EXAMPLE 48

1-/3-(5-Bromo-2,2-dimethyl-2,3-dihydrobenzo-furan-7-yloxy)-2-hydroxypropyl/-4-(3-trifluoro-methylphenyl)-1,2,3,6-tetrahydropyridine hydrochloride

To a solution of 2.63 g (0.0088 moles) of 5-bromo-2,2-dimethyl-7-oxiranylmethoxy-2,3-dihydrobenzofuran in 15 ml of ethanol, 2.11 g (0.008 moles) of 4-(3-trifluoromethyl-phenyl)-1,2,3,6-tetrahydropyridine hydro-chloride and 3.2 ml of 10% aqueous sodium hydroxide are added. The reaction mixture is boiled for 6 hours under stirring, then evaporated under reduced pressure. The residue is subjected to partition between 50 ml of dichloromethane and 50 ml of water. The organic phase is extracted twice with 20 ml of water each time, dried over anhydrous sodium sulfate, filtered, and evaporated under reduced pressure to remove the solvent. The residue is dissolved in 10 ml of ethanol containing 10% of hydrogen chloride, and the solution obtained is also evaporated to dryness under reduced pressure. The crystalline residue is boiled with 50 ml of ethyl acetate, the crystals are filtered from the hot mixture, and dried under reduced pressure.

Thus, 3.78 g (89.8%) of the title compound are obtained. M.p.: 112-114° C.

### EXAMPLE 49

1-/3-(5-Bromo-2,2-dimethyl-2,3-dihydrobenzo-furan-7-yloxy)-2-hydroxypropyl/-4-(3-trifluoro-methylphenyl)-1,2,3,6-tetrahydropyridine hydrochloride

To a solution of 2.30 g (0.0077 moles) of 5-bromo-2,2-dimethyl-7-oxiranylmethoxy-2,3-dihydrobenzofuran in 15 ml of ethanol, 1.72 g (0.007 moles) of 4-(3-trifluoromethylphenyl)-1,2,3,6-tetrahydropyridine are added, and the reaction mixture is boiled for 6 hours under stirring, then evaporated under reduced pressure. The residue is boiled in 10 ml of ethanol containing 20% of hydrogen chloride for 30 minutes, and the mixture is also evaporated under reduced pressure. The crystalline residue is boiled with 30 ml of ethyl acetate for 5 minutes, the crystals are filtered, and dried under reduced pressure.

Thus, 3.42 g (92.8%) of the title compound are obtained. M.p.: 112-114° C.

### EXAMPLE 50

1-/3-(2,2-Dimethyl-2,3-dihydrobenzofuran-7-yloxy)-2-hydroxypropyl/-4-(3-trifluoromethyl-phenyl)piperidine hydrochloride

A solution of 1.78 g (0.004 moles) of 1-3-(2,2-dimethyl-2,3-dihydrobenzofuran-7-yloxy)-2-hydroxypropyl/-4-(3-trifluoromethyl-phenyl)-1,2,3,6-tetrahydropyridine hydro-chloride in 20 ml of methanol is hydrogenized in the presence of 0.1 g of 10% palladium/carbon catalyst at 20° C. and atmospheric pressure. As soon as the calculated amount of hydrogen (96 ml) is taken up, the catalyst is removed by filtration, and the solution is evaporated under reduced pressure. The crystalline residue is rubbed with 20 ml of ether, the crystals are filtered, and dried under reduced pressure.

Thus, 1.75 g (98%) of the title compound are obtained. M.p.: 172-174° C.

### EXAMPLE 51

1-/3-(2,2-Dimethyl-2,3-dihydrobenzofuran-7-yloxy)-2-hydroxypropyl/-4-(3-trifluoromethyl-phenyl)piperidine hydrochloride

A mixture of 2.31 g (0.0105 moles) of 2,2-dimethyl-7-oxiranylmethoxy-2,3-dihydro-benzofuran and 2.45 g (0.01 moles) of 4-hydroxy-4-(3-trifluoromethylphenyl) piperidine is melted at 80° C. for 1 hour, then dissolved in 10 ml of ethanol containing 20% of hydrogen chloride, and the mixture is boiled for 45 minutes. The solution is diluted with 10 ml of ethanol, cooled to 30° C., 0.1 g of 10% palladium/carbon catalyst are added, and the mixture is hydrogenized. As soon as the calculated amount of hydrogen (240 ml) is taken up, the catalyst is removed by filtration, and the solution is evaporated under reduced pressure. The crystalline residue is rubbed with ether, the crystals are filtered, and dried under reduced pressure.

Thus, 4.05 g (91%) of the title compound are obtained. M.p.: 172-174° C.

### EXAMPLE 52

1-/3-(2,2-Dimethyl-2,3-dihydrobenzofuran-7-yloxy)-2-hydroxypropyl/-4-hydroxypiperidine hydrochloride

To a solution of 1.32 g (0.006 moles) of 2,2-dimethyl-7-oxiranylmethoxy-2,3-dihydro-benzofuran in 10 ml of tetrahydrofuran, 0.55 g (0.0055 moles) of 4-hydroxypiperidine are added. The reaction mixture is boiled for 3 hours under stirring, then evaporated to dryness under reduced pressure. The residue is dissolved in a mixture of 5 ml of 2-propanol and 1.5 ml of 2-propanol containing 16% of hydrogen chloride, the solution obtained is evaporated under reduced pressure, the residue is dissolved in 8 ml of 2-propanol, and allowed to stand at 0° C. for 5 days. The crystals precipitated are filtered, recrystallized from 2-propanol, filtered, and dried under reduced pressure.

Thus, 1.22 g (57%) of the title compound are obtained. M.p.: 139-141° C.

### EXAMPLE 53

1-/3-(2,2-Dimethyl-2,3-dihydrobenzofuran-7-yloxy)-2-hydroxypropyl/-4-methylpiperidine hydrochloride

A mixture of 1.32 g (0.006 moles) of 2,2-dimethyl-7-oxiranylmethoxy-2,3-dihydro-benzofuran, 0.44 g (0.0045 moles) of 4-methyl-piperidine and 8 ml of water is boiled for 9 hours. The oillike phase of the mixture formed is dissolved in 20 ml of ether, washed with water, dried over anhydrous sodium sulfate, evaporated under reduced pressure, dissolved in 3 ml of 2-propanol containing 16% of hydrogen chloride, and evaporated under reduced pressure. The residue is dissolved in ethyl acetate, precipitated with ether, the crystals are filtered, dissolved in hot 2-propanol, precipitated again with ether, the crystals are filtered, and dried under reduced pressure.

Thus, 1.25 g (59%) of the title compound are obtained. M.p.: 146-148° C.

### EXAMPLE 54

1-/3-(2,2-Dimethyl-2,3-dihydrobenzofuran-4-yloxy)-2-hydroxypropyl/-4-hydroxy-4-(4-tri-fluoromethylphenyl)piperidine hydrochloride

3.28 g (0.02 moles) of 2,2-dimethyl-2,3-dihydrobenzofuran-4-ol are dissolved in 25 ml of 10% aqueous sodium hydroxide. To the solution, 3.70 g (0.04 moles) of epichlorohydrin are added, and the reaction mixture is stirred at 45 to 50° C. for 3 hours. After cooling, the oily product that separates is dissolved in 30 ml of dichloromethane, the phases are separated, the organic phase is washed twice with 20 ml of water each time, dried over anhydrous sodium sulfate, filtered, and evaporated to dryness under reduced pressure. The residual thick honeylike epoxide (3.78 g, 85.3%) is dissolved in 40 ml of ethanol, to the solution obtained, 3.80 g (0.0155 moles) of 4-hydroxy-4-(3-trifluoro-methylphenyl)piperidine are added, and the reaction mixture is boiled for 6 hours. After cooling, to the solution obtained, 15 ml of ethanol containing 5% of hydrogen chloride are added at a temperature of less than 15° C., and the mixture is evaporated to dryness under reduced pressure. The crystalline residue is recrystallized from a mixture of ethanol and ether, filtered, washed with ether.

Thus, 5.60 g (72%) of the title compound are obtained. M.p.: 162-164° C.

### EXAMPLE 55

1-/3-(2,2-Dimethyl-2,3-dihydrobenzofuran-4-yloxy)-2-hydroxypropyl/-4-hydroxy-4-(6-methoxynaphth-2-yl)piperidine hydrochloride

The procedure of Example 54 is followed with the difference that the epoxide obtained in the first reaction step (3.75 g, 85.2%) is dissolved in 50 ml of ethanol. To the solution, 3.94 g (0.0153 moles) of 4-hydroxy-4-(6-methoxy-naphth-2-yl)piperidine are added, and the reaction mixture is boiled for 4 hours. After cooling, to the solution, 13 ml of ethanol containing 5% of hydrogen chloride are added, and the reaction mixture is evaporated to dryness under reduced pressure. The residual crystalline product is recrystallized from a mixture of ethanol and ether.

Thus, 5.35 g (68%) of the title compound are obtained. M.p.: 118-120° C.

### EXAMPLE 56

1-/3-(2,2-Dimethyl-2,3-dihydrobenzofuran-5-yloxy)-2-hydroxypropyl/-4-hydroxy-4-(4-tri-fluoromethylphenyl)piperidine hydrochloride

3.28 g (0.02 moles) of 2,2-dimethyl-2,3-dihydrobenzofuran-5-ol are dissolved in 30 ml of aqueous solution containing 3 g of sodium hydroxide. To the solution obtained, 3.70 g (0.04 moles) of epichloro-hydrin are added, and the reaction mixture is stirred at 48 to 50° C. for 3 hours. After cooling, the oily product that separates is dissolved in 30 ml of dichloromethane, the phases are separated, the organic phase is washed twice with 20 ml of water each time, dried over anhydrous sodium sulfate, filtered, and evaporated to dryness under reduced pressure. The residual thick waxlike epoxide (3.85 g, 87.3%) is dissolved in 50 ml of ethanol, to the solution, 3.80 g (0.0155 moles) of 4-hydroxy-4-(trifluoromethylphenyl)-piperidine are added, and the reaction mixture is boiled for 5 hours. After cooling, to the solution obtained, 15 ml of ethanol containing 5% of hydrogen chloride are added at a temperature of less than 15° C., and the mixture is evaporated to dryness under reduced pressure. The residue that crystallizes is recrystallized from a mixture of ethanol and ether, filtered, and washed with ether.

Thus, 4.97 g (64%) of the title compound are obtained. M.p.: 172-173° C.

### EXAMPLE 57

1-/3-(2,2-Dimethyl-2,3-dihydrobenzofuran-6-yloxy)-2-hydroxypropyl/-4-hydroxy-4-phenyl-piperidine hydrochloride

1.64 g (0.01 moles) of 2,2-dimethyl-2,3-dihydro-benzofuran-6-ol are dissolved in 15 ml of 10% aqueous sodium hydroxide. To the solution, 1.85 g (0.02 moles) of epichlorohydrin are added, and the reaction mixture is stirred at 48 to 52° C. for 2.5 hours. After cooling, the oily product that separates is dissolved in 25 ml of dichloro-methane, the phases are separated, the organic phase is washed twice with 15 ml of water each time, dried over anhydrous sodium sulfate, filtered, and evaporated to dryness under reduced pressure. The residual thick honeylike epoxide (2.10 g, 95%) is dissolved in 10 ml of ethanol, to the solution obtained, 1.52 g (0.0086 moles) of 4-hydroxy-4-phenyl-piperidine are added, and the reaction mixture is boiled for 6 hours. After cooling, to the solution obtained, 10 ml of ethanol containing 5% of hydrogen chloride are added at a temperature of less than 15° C., and the mixture is evaporated to dryness under reduced pressure. The residue that crystallizes is recrystallized from a mixture of ethanol and ether, filtered, and washed with ether.

Thus, 4.97 g (64%) of the title compound are obtained. M.p.: 184-186° C.

### EXAMPLE 58

1-/3-(2,2-Dimethyl-2,3-dihydrobenzofuran-7-yloxy)-2-hydroxypropyl/-4-methoxy-4-(3-tri-fluoromethylphenyl)piperidine hydrochloride

To a solution of 0.20 g (0.005 moles) of sodium hydroxide in 15 ml of isopropanol, 1.21 g (0.0055 moles) of 2,2-dimethyl-7-oxiranylmethoxy-2,3-dihydrobenzofuran and 1.48 g (0.005 moles) of 4-methoxy-4-(3-tri-fluoromethylphenyl)piperidine hydrochloride are added. The reaction mixture is boiled for 6 hours under stirring, then evaporated to dryness under reduced pressure. The residue is subjected to partition between 50 ml of water and 50 ml of dichloromethane, the phases are separated, the organic phase is extracted twice with 20 ml of water each time, dried over anhydrous sodium sulfate, filtered, and evaporated to dryness under reduced pressure. The residue is dissolved in 50 ml of ether, and, to the solution, 1 ml of ethanol containing 20% of hydrogen chloride is added under cooling, the crystals precipitated are filtered, washed with ether, and dried under reduced pressure.

Thus, 1.62 g (62.8%) of the title compound are obtained. M.p.: 192-195° C.

### EXAMPLE 59

1-/3-(2,2-Dimethyl-2,3-dihydrobenzofuran-7-yloxy)-2-hydroxypropyl/-4-(diphenylmethyl)-piperazine

A solution of 2.20 g (0.01 moles) of 2,2-dimethyl-7-oxiranylmethoxy-2,3-dihydrobenzo furan and 2.52 g (0.01 moles) of 1-(diphenyl-methyl)piperazine in 30 ml of isopropanol is boiled for 6 hours. The solution is evaporated to dryness under reduced pressure, the residual product is rubbed with n-hexane, and filtered. The thus-obtained crude product (4.53 g) is recrystallized from 25 ml of ethanol, filtered, and dried under reduced pressure.

Thus, 3.89 g (82) of the title compound are obtaimed. M.p.: 129-130° C.

### EXAMPLE 60

1-/3-(2,2-Dimethyl-2,3-dihydrobenzofuran-7-yloxy)-2-hydroxypropyl/-4-(diphenylmethyl)-piperazine

A mixture of 2.20 g (0.01 moles) of 2,2-dimethyl-7-oxiranylmethoxy-2,3-dihydro-benzofuran and 2.52 g (0.01 moles) of 1-(diphenylmethyl)piperazine is melted at 80° C. and maintained at the above temperature for an hour. The obtained mass that solidifies is recrystallized from 25 ml of ethanol, filtered, and dried under reduced pressure.

Thus, 3.92 g (83%) of the title compound are obtained. M.p.: 129-130° C.

### EXAMPLE 61

1-/3-(2,2-Dimethyl-2,3-dihydrobenzofuran-7-yloxy)-2-hydroxypropyl/-4-(diphenylmethyl)-piperazine

A mixture of 2.20 g (0.01 moles) of 2,2-dimethyl-7-oxiranylmethoxy-2,3-dihydro-benzofuran and 2.52 g (0.01 moles) of 1-(diphenylmethyl)piperazine is melted at 60° C. and maintained at the above temperature for an hour. The obtained mass that solidifies is recrystallized from 25 ml of ethanol, filtered, and dried under reduced pressure.

Thus, 3.82 g (81%) of the title compound are obtained. M.p.: 129-130° C.

### EXAMPLE 62

1-/3-(2,2-Dimethyl-2,3-dihydrobenzofuran-7-yloxy)-2-hydroxypropyl/-4-(diphenylmethyl)-piperazine

To a solution of 2.31 g (0.011 moles) of 2,2-dimethyl-7-oxiranylmethoxy-2,3-dihydro-benzofuran in 20 ml of ethanol, 2.52 g (0.01 moles) of 1-(diphenylmethyl)piperazine are added, and the reaction mixture is boiled for 4 hours. After cooling, the crystals precipitated are filtered, and dried under reduced pressure.

Thus, 3.87 g (81%) of the title compound are obtained. M.p.: 129-130° C.

### EXAMPLE 63

1-/3-(2,2-Dimethyl-2,3-dihydrobenzofuran-7-yloxy)-2-hydroxypropyl/-4-(4-fluorophenyl)-piperazine

A solution of 4.40 g (0.02 moles) of 2,2-dimethyl-7-oxiranylmethoxy-2,3-dihydro-benzofuran and 3.60 g (0.02 moles) of 1-(4-fluorophenyl)piperazine in 30 ml of ethanol is boiled for 5 hours. The solution is evaporated to dryness under reduced pressure, the residual product is subjected to chromatography on a column filled with Kieselgel 60 using a mixture of 30 volumes of chloroform and 1 volume of ethanol as the eluent. The fractions containing the product are evaporated, the residual product is rubbed with n-hexane, and filtered, dried under reduced pressure.

Thus, 7.30 g (91%) of the title compound are obtained. M.p.: 80-82° C.

### EXAMPLE 64

1-/3-(2,2-Dimethyl-2,3-dihydrobenzofuran-7-yloxy)-2-hydroxypropyl/-4-(4-fluorophenyl)-piperazine

A mixture of 2.31 g (0.011 moles) of 2,2-dimethyl-7-oxiranylmethoxy-2,3-dihydrobenzo-furan and 1.80 g (0.01 moles) of 1-(4-fluoro-phenyl)piperazine is melted at 60° C., and maintained at the above temperature for an hour. The melt obtained is subjected to chromatography on a column filled with Kieselgel 60 using a mixture consisting og 30 volumes of chloroform and 1 volume of ethanol as the eluent. The fractions containing the product are evaporated, and the residue is rubbed with n-hexane, then filtered, and dried under reduced pressure.

Thus, 3.72 g (93%) of the title compound are obtained. M.p.: 80-82° C.

### EXAMPLE 65

1-/3-(2,2-Dimethyl-2,3-dihydrobenzofuran-7-yloxy)-2-hydroxypropyl/-4-(4-fluorophenyl)-piperazine

A mixture of 2.20 g (0.011 moles) of 2,2-dimethyl-7-oxiranylmethoxy-2,3-dihydro-benzofuran and 1.80 g (0.01 moles) of 1-(4-fluorophenyl)piperazine is melted at 70° C. and maintained at the latter temperature for 1 hour. To the melt obtained, 60 ml of n-hexane are added, the mixture is cooled, the crystals precipitated are filtered, dried under reduced pressure.

Thus, 3.26 g (81%) of the title compound are obtained. M.p.: 80-82° C.

### EXAMPLE 66

1-/3-(2,2-Dimethyl-2,3-dihydrobenzofuran-7-yloxy)-2-hydroxypropyl/-4-(3-fluorophenyl)-piperazine

A solution of 2.20 g (0.01 moles) of 2,2-dimethyl-7-oxiranylmethoxy-2,3-dihydrobenzo-furan and 2.30 g (0.01 moles) of 1-(3-tri-fluoromethylphenyl)piperazine in 20 ml of isopropanol is boiled for 4 hours. The solution is evaporated to dryness under reduced pressure, the residue is subjected to chromatography on a column filled with Kieselgel 60 and using a mixture of 30 volumes of chloroform and 1 volume of ethanol as the eluent. The fractions containing the product are evaporated, the product obtained is rubbed with n-hexane, filtered, and dried under reduced pressure.

Thus, 3.76 g (84%) of the title compound are obtained. M.p.: 82-84° C.

### EXAMPLE 67

1-/3-(2,2-Dimethyl-2,3-dihydrobenzofuran-7-yloxy)-2-hydroxypropyl/-4-(3-fluorophenyl)-piperazine

A mixture of 2.20 g (0.01 moles) of 2,2-dimethyl-7-oxiranylmethoxy-2.3-dihydrobenzo-furan and 2.30 g (0.01 moles) of 1-(3-tri-fluoromethylphenyl)piperazine is melted at 70° C., and maintained at the latter temperature for 1.5 hours. To the melt obtained, 60 ml of n-hexane are added, the mixture is cooled, the crystals precipitated are filtered, and dried under reduced pressure.

Thus, 3.77 g (84%) of the title compound are obtained. M.p.: 82-84° C.

### EXAMPLE 68

1-/3-(2,2-Dimethyl-2,3-dihydrobenzofuran-7-yloxy)-2-hydroxypropyl/-4-(4-methoxyphenyl)-piperazine

A solution of 3.80 g (0.017 moles) of 2,2-dimethyl-7-oxiranylmethoxy-2,3-dihydro-benzofuran and 2.88 g (0.015 moles) of 1-(4-methoxyphenyl)piperazine in 40 ml of methyl tert.-butyl ether is boiled for 8 hours, then cooled to 0° C. The crystals precipitated are filtered, and dried under reduced pressure.

Thus, 5.21 g (84%) of the title compound are obtained. M.p.: 93-94° C.

### EXAMPLE 69

1-/3-(2,2-Dimethyl-2,3-dihydrobenzofuran-7-yloxy)-2-hydroxypropyl/-4-(4-methoxyphenyl)-piperazine

A mixture of 1.10 g (0.005 moles) of 2,2-dimethyl-7-oxiranylmethoxy-2,3-dihydro-benzofuran and 0.96 g (0.005 moles) of 1-(4-methoxyphenyl)piperazine is melted at 80° C., and maintained at the latter temperature for 1 hour. To the warm melt obtained, 10 ml of methyl tert.-butyl ether are added, the mixture is cooled, the crystals precipitated are filtered, and dried under reduced pressure.

Thus, 1.88 g (91%) of the title compound are obtained. M.p.: 93-94° C.

### EXAMPLE 70

1-/3-(2,2-Dimethyl-2,3-dihydrobenzofuran-7-yloxy)-2-hydroxypropyl/-4-(benzo-1,3-dioxolane-5-yl)piperazine dihydrochloride

A solution of 3.80 g (0.017 moles) of 2,2-dimethyl-7-oxiranylmethoxy-2,3-dihydro-benzofuran and. 3.30 g (0.015 moles) of 1-(benzo-1,3-dioxolane-5-yl)piperazine in 30 ml of ethanol is boiled for 10 hours. The solution is evaporated to dryness under reduced pressure, the residue (8.0 g) is dissolved in 30 ml of ethanol containing 4.0 g of hydrogen chloride. From the homogeneous solution, crystals begin to separate. The suspension is diluted with 60 ml of methyl tert.-butyl ether, filtered, and the crystals are dried under reduced pressure.

Thus, 5.40 g (70%) of the title compound are obtained. M.p.: 216-218° C.

### EXAMPLE 71

1-/3-(2,2-Dimethyl-2,3-dihydrobenzofuran-7-yloxy)-2-hydroxypropyl/-4-(benzo-1,3-dioxolane-5-yl)piperazine dihydrochloride

A solution of 3.80 g (0.017 moles) of 2,2-dimethyl-7-oxiranylmethoxy-2,3-dihydro-benzofuran and 3.30 g (0.015 moles) of 1-(benzo-1,3-dioxolane-5-yl)piperazine in 30 ml of isopropanol is boiled for 6 hours. The solution is evaporated to dryness under reduced pressure, the residue is dissolved in 20 ml of ethanol containing 15% of hydrogen chloride. From the homogeneous solution, crystals begin to separate. The suspension is diluted with 60 ml of methyl tert.-butyl ether, filtered, and the crystals are dried under reduced pressure.

Thus, 5.43 g (71%) of the title compound are obtained. M.p.: 216-218° C.

### EXAMPLE 72

1-/3-(2,2-Dimethyl-2,3-dihydrobenzofuran-7-yloxy)-2-hydroxypropyl/-4-(benzo-1,3-dioxolane-5-yl)piperazine dihydrochloride

A solution of 2.31 g (0.011 moles) of 2,2-dimethyl-7-oxiranylmethoxy-2,3-dihydro-benzofuran and 2.20 g (0.01 moles) of 1-(benzo-1,3-dioxolane-5-yl)piperazine in 30 ml of diisopropyl ether is boiled for 6 hours, then, 7 ml of ethanol containing 20% of hydrogen chloride are added. The crystals precipitated are cooled, filtered, and dried under reduced pressure.

Thus, 4.41 g (86%) of the title compound are obtained. M.p.: 216-218° C.

### EXAMPLE 73

1-/3-(2,2-Dimethyl-2,3-dihydrobenzofuran-7-yloxy)-2-hydroxypropyl/-4-(4-chlorophenyl)-piperazine

A mixture of 2.42 g (0.0115 moles) of 2,2-dimethyl-7-oxiranylmethoxy-2,3-dihydro-benzofuran, 2.70 g (0.01 moles) of 1-(4-chloro-phenyl)piperazine dihydrochloride, 26 ml of ethanol, 3 ml of water and 0.9 g (0.0225 moles) of sodium hydroxide is boiled for 6 hours under stirring, then evaporated to dryness under reduced pressure. The residue is subjected to partition between 80 ml of dichloromethane and 50 ml of water, the organic phase is extracted twice using 30 ml of water each time, dried over anhydrous sodium sulfate, filtered, and evaporated to dryness under reduced pressure. The residue is recrystallized from 8 ml of methanol, the crystals precipitated are filtered, and dried under reduced pressure.

Thus, 3.25 g (78%) of the title compound are obtained. M.p.: 96-98° C.

### EXAMPLE 74

1-/3-(2,2-Dimethyl-2,3-dihydrobenzofuran-7-yloxy)-2-hydroxypropyl/-4-(4-chlorophenyl)-piperazine

To a solution of 2.70 g (0.01 moles) of 1-(4-chlorophenyl)piperazine dihydrochloride in 26 ml of ethanol, 3 ml of water and 0.9 g (0.0225 moles) of sodium hydroxide are added, and the mixture is boiled for 10 minutes. Then, to the reaction mixture, 2.40 g (0.011 moles) of 2,2-dimethyl-7-oxiranylmethoxy-2,3-dihydrobenzofuran are added under stirring, and the mixture is boiled for further 4 hours, then evaporated to dryness under reduced pressure. To the residue, 50 ml of water are added, the water above the substance that slowly hardens is decanted, and the residue is rubbed with a further portion of water. The crystals precipitated are filtered, washed with water, recrystallized from methanol, filtered, and dried under reduced pressure.

Thus, 3.17 g (80%) of the title compound are obtained. M.p.: 96-98° C.

### EXAMPLE 75

1-/3-(2,2-Dimethyl-2,3-dihydrobenzofuran-7-yloxy)-2-hydroxypropyl/-4-(4-chlorophenyl)-piperazine dihydrochloride

To a solution of 0.40 g (0.001 moles) of 1-/3-(2,2-dimethyl-2,3-dihydrobenzofuran-7-yloxy)-2-hydroxypropyl/-4-(4-chlorophenyl)-piperazine in 5 ml of ethanol, 1 ml of ethanol containing 20% of hydrogen chloride is added, and the mixture is evaporated to dryness under reduced pressure. The residue is boiled with 5 ml of ethyl acetate for 5 minutes, the hot suspension is filtered, and the crystals are dried under reduced pressure.

Thus, 0.43 g (91%) of the title compound are obtained. M.p.: 168-170° C.

### EXAMPLE 76

1-/3-(2,2-Dimethyl-2,3-dihydrobenzofuran-7-yloxy)-2-hydroxypropyl/-4-(3-methoxyphenyl)-piperazine dihydrochloride

A mixture of 1.32 g (0.006 moles) of 2,2-dimethyl-7-oxiranylmethoxy-2,3-dihydro-benzofuran, 1.46 g (0.0055 moles) of 1-(3-methoxyphenyl)piperazine dihydrochloride, 0.44 g (0.011 moles) of sodium hydroxide, 5 ml of ethanol, 3 ml of dimethylformamide and 10 ml of water is boiled for 3 hours. The reaction mixture is cooled to 20° C., the phases are separated, the lower (organic) phase is dissolved in 20 ml of ether, dried over anhydrous sodium sulfate, and evaporated to dryness under reduced pressure. The residue is dissolved in 3 ml of 2-propanol, to the solution formed, 3 ml of 2-propanol containing 16% of hydrogen chloride are added, and the reaction mixture is maintained at 0° C. for 5 days. The crystals precipitated are filtered, recrystallized from 2-propanol, filtered again, and dried under reduced pressure.

Thus, 1.48 g (60%) of the title compound are obtained. M.p.: 168-170° C.

### EXAMPLE 77

1-/3-(2,2-Dimethyl-2,3-dihydrobenzofuran-7-yloxy)-2-hydroxypropyl/-4-benzylpiperazine dihydrochloride

A mixture of 1.32 g (0.006 moles) of 2,2-dimethyl-7-oxiranylmethoxy-2,3-dihydro-benzofuran, 0.97 g (0.0055 moles) of 1-benzyl-piperazine and 8 ml of 2-propanol is boiled for 4 hours, then cooled to 20° C. To the reaction mixture, 20 ml of petroleum ether are added, the mixture is maintained at 0° C. for 5 days, the crystals formed are filtered, then recrystallized from n-hexane. The crystals precipitated are dissolved in 15 ml of 2-propanol, and, to the solution obtained, 1.5 ml of 2-propanol containing 16% of hydrogen chloride are added. After cooling, the crystalline salt precipitated is filtered, and dried under reduced pressure.

Thus, 1.50 g (58%) of the title compound are obtained. M.p.: 188-191° C.

### EXAMPLE 78

1-/3-(2,2-Dimethyl-2,3-dihydrobenzofuran-7-yloxy)-2-hydroxypropyl/-4-(2,4-dichloro-phenyl)piperazine hydrochloride

A mixture of 1.32 g (0.006 moles) of 2,2-dimethyl-7-oxiranylmethoxy-2,3-dihydro-benzofuran, 1.15 g (0.005 moles) of 1-(2,4-dichlorophenyl)piperazine and 8 ml of 2-propanol is boiled for 5 hours, then cooled to 20° C. To the reaction mixture, 3.6 ml of 2-propanol and 2.4 ml of 2-propanol containing 16% of hydrogen chloride are added, and the mixture is stirred at room temperature for 5 hours. The crystals precipitated are filtered, recrystallized from 2-propanol, filtered, then dried under reduced pressure.

Thus, 1.19 g (45%) of the title compound are obtained. M.p.: 169-171° C.

### EXAMPLE 79

1-/3-(2,2-Dimethyl-2,3-dihydrobenzofuran-7-yloxy)-2-hydroxypropyl/-4-(3-chlorophenyl)-piperazine hydrochloride

A mixture of 2.64 g (0.012 moles) of 2,2-dimethyl-7-oxiranylmethoxy-2,3-dihydro-benzofuran, 2.36 g (0.012 moles) of 1-(3-chlorophenyl)piperazine and 16 ml of 2-propanol is boiled for 3 hours. After cooling, to the solution obtained, 7 ml of 2-propanol containing 16% of hydrogen chloride are added at 20° C. The reaction mixture is maintained at 0° C. for 2 days, the crystals precipitated are filtered, recrystallized from 2-propanol, filtered again, and dried under reduced pressure.

Thus, 2.88 g (53%) of the title compound are obtained. M.p.: 187-190° C.

### EXAMPLE 80

1-/3-(2,2-Dimethyl-2,3-dihydrobenzofuran-7-yloxy)-2-hydroxypropyl/-4-(2-pyridyl)-piperazine trihydrochloride

A mixture of 1.32 g (0.006 moles) of 2,2-dimethyl-7-oxiranylmethoxy-2,3-dihydro-benzofuran, 0.78 g (0.0048 moles) of 1-(2-pyridyl)piperazine and 8 ml of petroleum ether is boiled for 6 hours. After cooling to 20° C., the phases that form are separated, to the lower phase, 20 ml of 2-propanol and 4.5 ml of 2-propanol containing 16% of hydrogen chloride are added, and the reaction mixture is maintained at 0° C. for 5 days. The crystals separated are filtered, recrystallized from 2-propanol, filtered, and dried under reduced pressure.

Thus, 1.68 g (71%) of the title compound are obtained. M.p.: 133-136° C.

### EXAMPLE 81

1-/3-(2,2-Dimethyl-2,3-dihydrobenzofuran-7-yloxy)-2-hydroxypropyl/-4-(2-methoxyphenyl)-piperazine dihydrochloride

A mixture of 2.64 g (0.012 moles) of 2,2-dimethyl-7-oxiranylmethoxy-2,3-dihydro-benzofuran, 2.36 g (0.011 moles) of 1-(2-methoxyphenyl)piperazine and 16 ml of 2-propanol is boiled for 5.5 hours. After cooling to 20° C., to the reaction mixture, 10 ml of 2-propanol and 8 ml of 2-propanol containing 16% of hydrogen chloride are added, and the reaction mixture is maintained at 0° C. for 2 days. The crystals precipitated are filtered using reduced pressure, washed with 2-propanol, recrystallized from 2-propanol, filtered again, and dried under reduced pressure.

Thus, 2.53 g (47%) of the title compound are obtained. M.p.: 128-130° C.

### EXAMPLE 82

1-/3-(2,2-Dimethyl-2,3-dihydrobenzofuran-7-yloxy)-2-hydroxypropyl/-4-(3,4-dimethyl-phenyl)piperazine dihydrochloride

A mixture of 1.32 g (0.006 moles) of 2,2-dimethyl-7-oxiranylmethoxy-2,3-dihydro-benzofuran, 0.95 g (0.005 moles) of 1-(3,4-dimethylphenyl)piperazine and 8 ml of ethanol is boiled for 6 hours. After cooling to 20° C., the reaction mixture is filtered, and the filtrate is evaporated to dryness under reduced pressure. To the residue, 5 ml of 2-propanol containing 16% of hydrogen chloride are added, the reaction mixture is maintained at 0° C. for 2 days, the crystals precipitated are filtered, recrystallized from 2-propanol, filtered again, and dried under reduced pressure.

Thus, 1.50 g (62%) of the title compound are obtained. M.p.: 119-122° C.

### EXAMPLE 83

1-/3-(2,2-Dimethyl-2,3-dihydrobenzofuran-7-yloxy)-2-hydroxypropyl/-4-(2-pyrimidyl)-piperazine dihydrochloride

A mixture of 1.32 g (0.006 moles) of 2,2-dimethyl-7-oxiranylmethoxy-2,3-dihydro-benzofuran, 0.65 g (0.004 moles) of 2-pyrimidylpiperazine and 8 ml of tetrahydro-furan is boiled for 12 hours, then cooled to 20° C. To the oil that forms, 20 ml of ether and 1.5 ml of 2-propanol containing 30% of hydrogen chloride are added, the reaction mixture is maintained at 0° C. for 5 days, the crystals precipitated are filtered, dried under reduced pressure, and recrystallized from 2-propanol.

Thus, 1.50 g (82%) of the title compound are obtained. M.p.: 128-130° C.

### EXAMPLE 84

1-/3-(2,2-Dimethyl-2,3-dihydrobenzofuran-7-yloxy)-2-hydroxypropyl/-4-(4-chloro-2-methyl-phenyl)piperazine hydrochloride

A mixture of 2.64 g (0.012 moles) of 2,2-dimethyl-7-oxiranylmethoxy-2,3-dihydro-benzofuran, 2.27 g (0.008 moles) of 1-(4-chloro-2-methylphenyl)piperazine hydro-chloride, 0.64 g (0.016 moles) of sodium hydroxide and 16 ml of water is boiled for 3 hours, then cooled to 20° C. The aqueous phase is decanted, to the organic phase, 30 ml of ether are added, and stirred for an hour. The crystals precipitated are filtered, dried under reduced pressure, and recrystallized from acetonitrile.

Thus, 2.07 g (60%) of the title compound are obtained. M.p.: 68-70° C.

### EXAMPLE 85

1-/3-(2,2-Dimethyl-2,3-dihydrobenzofuran-7-yloxy)-2-hydroxypropyl/-4-methylpiperazine dihydrochloride

A mixture of 1.32 g (0.006 moles) of 2,2-dimethyl-7-oxiranylmethoxy-2,3-dihydro-benzofuran, 0.45 g (0.0045 moles) of 4-methyl-piperazine and 8 ml of ethanol is boiled for 3 hours, then cooled to 20° C. To the mixture, 4 ml of 2-propanol containing 30% of hydrogen chloride are added, the reaction mixture is maintained at −15° C. for 2 days, the crystals precipitated are filtered, dried under reduced pressure, dissolved in hot 2-propanol, and precipitated from the solution by diethyl ether.

Thus, 1.15 g (65%) of the title compound are obtained. M.p.: 119-122° C.

### EXAMPLE 86

1-/3-(2,2-Dimethyl-2,3-dihydrobenzofuran-7-yloxy)-2-hydroxypropyl/-4-(trifluoromethyl-benzyl)piperazine dihydrochloride

A mixture of 1.32 g (0.006 moles) of 2,2-dimethyl-7-oxiranylmethoxy-2,3-dihydro-benzofuran, 1.43 g (0.0045 moles) of 4-(3-trifluoromethyl)benzylpiperazine dihydro-chloride, 0.36 g (0.009 moles) of sodium hydroxide and 8 ml of water is boiled for 2.5 hours, then cooled to 20° C., the aqueous phase is removed by decantation, to the organic phase, 2 ml of 2-propanol containing 30% of hydrogen chloride are added, the crystals precipitated are filtered, dried under reduced pressure, and recrystallized from 2-propanol.

Thus, 1.39 g (57%) of the title compound are obtained. M.p.: 209-211° C.

### EXAMPLE 87

1-/3-(2,2-Dimethyl-2,3-dihydrobenzofuran-7-yloxy)-2-hydroxypropyl/-4-(3,4-dichloro-phenyl)piperazine hydrochloride

A mixture of 1.32 g (0.006 moles) of 2,2-dimethyl-7-oxiranylmethoxy-2,3-dihydro-benzofuran, 1.04 g (0.0045 moles) of 1-(3,4-dichlorophenyl)piperazine and 8 ml of 2-propanol is boiled for 3 hours, then cooled to 20° C. To the reaction mixture, a mixture of 10 ml of 2-propanol and 2 ml of 2-propanol containing 30% of hydrogen chloride are added, the reaction mixture is stirred for 5 hours, the crystals precipitated are filtered, dried under reduced pressure, and recrystallized from 2-propanol.

Thus, 1.63 g (62%) of the title compound are obtained. M.p.: 180-182° C.

### EXAMPLE 88

1-/3-(2,2-Dimethyl-2,3-dihydrobenzofuran-7-yloxy)-2-hydroxypropyl/-4-(2,6-dimethyl-phenyl)piperazine dihydrochloride

A mixture of 1.32 g (0.006 moles) of 2,2-dimethyl-7-oxiranylmethoxy-2,3-dihydro-benzofuran, 0.90 g (0.004 moles) of 1-(2,6-dimethylphenyl)piperazine hydrochloride, 0.32 g (0.008 moles) of sodium hydroxide and 8 ml of water is boiled for 2 hours, then cooled to 20° C. The aqueous phase of the mixture formed is removed by decantation, to the organic phase, 2.5 ml of 2-propanol containing 30% of hydrogen chloride are added, the reaction mixture is maintained at −15° C. for 2 days, then 10 ml of, 2-propanol are added, the crystals precipitated are filtered, dried under reduced pressure, and recrystallized from 2-propanol.

Thus, 1.00 g (41%) of the title compound are obtained. M.p.: 115-117° C.

### EXAMPLE

1-/3-(2,2-Dimethyl-2,3-dihydrobenzofuran-7-yloxy)-2-hydroxypropyl/-4-(4-chloro-2-methyl-phenyl)piperazine

A mixture of 0.5 g (0.0016 moles) of 1-bromo-3-(2,2-dimethyl-2,3-dihydrobenzofuran-7-yloxy)-2-propanol, 0.45 g (0.0016 moles) of 4-chloro-2-methylphenylpiperazine, 0.25 g (0.0063 moles) of sodium hydroxide and 6.5 ml of water is boiled for 3 hours, then cooled to 20° C. The aqueous phase is removed by decantation, the residual oil is rubbed with ether, the crystals precipitated are filtered, dried under reduced pressure, and recrystallized from acetonitrile.

Thus, 0.25 g (40%) of the title compound are obtained. M.p.: 68-70° C.

1. A novel benzofuran derivative of the formula 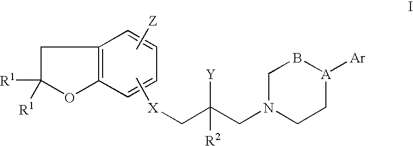
wherein
R¹ and R² represent, independently, a hydrogen atom or a C₁₋₄ alkyl group,
X stands for an oxygen atom or a sulfur atom,
Y means a hydrogen atom or a hydroxy group,
Z represents a hydrogen atom; a halo atom, a C₁₋₄ alkyl group, a C₁₋₄ alkoxy group, an amino group, a nitro group, a cyano group, a trifluoromethyl group, a group of the formula —COOR³, —NHCOR³ or —SO₂NR³R⁴, wherein
R³ stands for a hydrogen atom or a C₁₋₄ alkyl group,
R⁴ is a C₁₋₄ alkyl group, or
R³ and R⁴ form, together with the adjacent nitrogen atom, a saturated or unsaturated heterocyclic group having 5 to 10 members and optionally comprising one or more nitrogen atom(s) and/or one or more oxygen atom(s) and/or one or more sulfur atom(s) as the further heteroatom(s),
A means a group of the formula CH, COH, C—CN, C—COOR³ or COR⁴, wherein R³ and R⁴ are as defined above,
B represents a methylene group, or
A forms together with B a group of the formula —C═C—,
Ar stands for a hydrogen atom, a C₁₋₄ alkyl group, a phenyl(C₁₋₄ alkyl) group, a biphenylyl group, a naphthyl group, wherein said latter species are optionally substituted by a C₁₋₄ alkoxy group or a C₂₋₄ alkenyl group; a partially saturated, 5- or 6-membered heterocyclic group condensed with a phenyl group and containing one or two oxygen atom(s), said heterocyclic group being optionally substituted by one to three C₁₋₄ alkyl group; a 5- or 6-membered, saturated or unsaturated hetero cyclic group containing a nitrogen atom and/or an oxygen atom and/or a sulfur atom as the heteroatom; or a phenyl group substituted by the substituents R⁵, R⁶ and R⁷, wherein
R⁵, R⁶ and R⁷ mean, independently, a hydrogen atom, a halo atom, a trifluoro-methyl group, a C₁₋₄ alkyl group, a methylenedioxy group, a phenoxy group optionally substituted by a C₁₋₄ alkoxy group or by a halo atom; a C₂₋₄ alkenyl group, a C₂₋₄ alkenyloxy group, a C₁₋₄ alkoxy group optionally substituted by a di(C₁₋₄ alkyl)amino group or by a 5- or 6-membered, saturated hetero-cyclic group containing one or two nitrogen atom(s) or a nitrogen atom and an oxygen atom, wherein said heterocyclic group is optionally substituted by a C₁₋₄ alkyl group, or
A stands for a group of the formula N—(CH₂)ₙ—Ar′, wherein
Ar′ represents a diphenylmethyl group, a pyridyl group, a pyrimidinyl group, a naphthyl group, wherein said latter group is optionally substituted by a C₁₋₄ alkoxy group or a C₂₋₄ alkenyloxy group; a partially saturated, 5- or 6-membered heterocyclic group condensed with a phenyl group and containing one or two oxygen atom(s), said hetero-cyclic group being optionally substituted by one to three C₁₋₄ alkyl group(s); or a phenyl group substituted by the substituents R⁵, R⁶ and R⁷ wherein R⁵, R⁶ and R⁷ are as defined above,
n has a value of 0 or 1,
and pharmaceutically suitable acid addition salts thereof.

2. A benzofuran derivative as claimed in claim 1, wherein in formula I
R¹ represents a hydrogen atom or a C₁₋₄ alkyl group,
R² stands for a hydrogen atom,
X means an oxygen atom,
Y is a hydrogen atom or a hydroxy group,
Z represents a hydrogen atom, a halo atom or a nitro group,
A stands for a group of the formula CH, COH or C—CN,
B means a methylene group, or
A forms with B a group of the formula —C═C—,
Ar represents a hydrogen atom, a benzyl group, a phenyl group substituted by substituents R⁵, R⁶ and R⁷, a biphenylyl group, a naphthyl group optionally substituted by a C 4 alkoxy group; or a thienyl group, wherein
R⁵, R⁶ and R⁷ mean, independently, a hydrogen atom, a halo atom, a trifluoro-methyl group, a C₁₋₄ alkyl group, a C₁₋₄ alkoxy group, a C₂₋₄ alkenyloxy group, a phenoxy group or a methylenedioxy group,
and pharmaceutically suitable acid addition salts thereof.

3. A benzofuran derivative as claimed in claim 1 or 2, wherein in formula I
R¹ represents a methyl group,
R² stands for a hydrogen atom,
X means an oxygen atom,
Y is a hydroxy group,
Z represents a hydrogen atom,
A is a group of the formula CH, COH or C—CN,
B stands for a methylene group, or
A forms with B a group of the formula —C═C—,
Ar represents a phenyl group optionally substituted by a halo atom, a trifluoro-methyl group, a methyl group or a methoxy group; or a methoxynaphthyl group,
and pharmaceutically suitable acid addition salts thereof.

4. A piperazinylalkylbenzofuran derivative of the formula 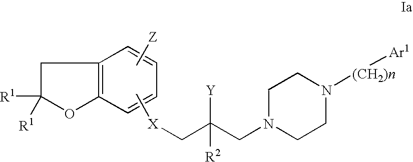
as claimed in claim 1, wherein
R¹ represents a C₁₋₄ alkyl group,
R² stands for a hydrogen atom,
X means an oxygen atom,
Y is a hydroxy group,
Z represents a hydrogen atom,
Ar′ represents a diphenylmethyl group, a pyridyl group, a partially saturated 5-membered heterocyclic group containing two oxygen atoms and being condensed with a phenyl group, or a phenyl group substituted by substituents R⁵, R⁶ and R⁷, wherein
R⁵, R⁶ and R⁷ mean, independently, a hydrogen atom, a halo atom, a trifluoro-methyl group, a C₁₋₄ alkyl group, a C₁₋₄ alkoxy group, or a methylenedioxy group,
n has a value of 0 or 1,
and pharmaceutically suitable acid addition salts thereof.

5. A piperazinylalkylbenzofuran derivative as claimed in claim 4, wherein in formula Ia
R¹ represents a methyl group,
R² stands for a hydrogen atom,
X means an oxygen atom,
Y is a hydroxy group,
Z represents a hydrogen atom,
Ar′ represents a diphenylmethyl group, a pyridyl group, a benzo-1,3-dioxolanyl group or a phenyl group optionally substituted by one or two halo atom(s), one or two methyl group(s), a methylenedioxy group, a trifluoromethyl group or a methoxy group,
n has a value of 0 or 1,
and pharmaceutically suitable acid addition salts thereof.

6. 1-/3-(2,2-dimethyl-2,3-dihydro-benzo-furan-7-yloxy)-2-hydroxypropyl/-4-(3-trifluoro-methylphenyl)-1,2,3,6-tetrahydropyridine, 1-/3-(2,2-dimethyl-2,3-dihydro-benzofuran-7-yl-oxy)-2-hydroxypropyl/-4-hydroxy-4-(3-trifluoro-methylphenyl)piperidine, 1-/3-(2,2-dimethyl-2,3-dihydro-benzofuran-7-yl-oxy)-2-hydroxypropyl/-4-hydroxy-4-(4-fluoro-phenyl)piperidine, 1-/3-(2,2-dimethyl-2,3-dihydro-benzofuran-7-yl-oxy)-2-hydroxypropyl/-4-hydroxy-4-phenyl-piperidine, 1-/3-(2,2-dimethyl-2,3-dihydro-benzofuran-7-yl-oxy)-2-hydroxypropyl/-4-hydroxy-4-(3-chloro-phenyl)piperidine, 1-/3-(2,2-dimethyl-2,3-dihydro-benzofuran-7-yl-oxy)-2-hydroxypropyl/-4-hydroxy-4-(3-methoxy-phenyl)piperidine, 1-/3-(2,2-dimethyl-2,3-dihydro-benzofuran-7-yl-oxy)-2-hydroxypropyl/-4-hydroxy-4-(4-methoxy-phenyl)piperidine, 1-/3-(2,2-dimethyl-2,3-dihydro-benzofuran-7-yl-oxy)-2-hydroxypropyl/-4-(3-trifluoromethyl-phenyl)piperidine, 1-/3-(2,2-dimethyl-2,3-dihydro-benzofuran-7-yl-oxy)-2-hydroxypropyl/-4-hydroxy-4-(4-methyl-phenyl)piperidine, 1-/3-(2,2-dimethyl-2,3-dihydro-benzofuran-7-yl-oxy)-2-hydroxypropyl/-4-cyano-4-phenyl-piperidine, 1-/3-(2,2-dimethyl-2,3-dihydro-benzofuran-7-yl-oxy)-2-hydroxypropyl/-4-hydroxy-4-(4-chloro-phenyl)piperidine, 1-/3-(2,2-dimethyl-2,3-dihydro-benzofuran-7-yl-oxy)-2-hydroxypropyl/-4-hydroxy-4-(6-methoxy-naphth-2-yl)piperidine, 1-/3-(2,2-dimethyl-2,3-dihydro-benzofuran-7-yl-oxy)-2-hydroxypropyl/-4-(diphenylmethyl)-piperazine, 1-/3-(2,2-dimethyl-2,3-dihydro-benzofuran-7-yl-oxy)-2-hydroxypropyl/-4-(4-fluorophenyl)-piperazine, 1-/3-(2,2-dimethyl-2,3-dihydro-benzofuran-7-yl-oxy)-2-hydroxypropyl/-4-hydroxy-4-(3-trifluoro-methylphenyl)piperazine, 1-/3-(2,2-dimethyl-2,3-dihydro-benzofuran-7-yl-oxy)-2-hydroxypropyl/-4-(4-methoxyphenyl)-piperazine, 1-/3-(2,2-dimethyl-2,3-dihydro-benzofuran-7-yl-oxy)-2-hydroxypropyl/-4-(benzo-1,3-dioxolan-5-yl)piperazine, 1-/3-(2,2-dimethyl-2,3-dihydro-benzofuran-7-yl-oxy)-2-hydroxypropyl/-4-(4-chlorophenyl)-piperazine, 1-/3-(2,2-dimethyl-2,3-dihydro-benzofuran-7-yl-oxy)-2-hydroxypropyl/-4-benzylpiperazine, 1-/3-(2,2-dimethyl-2,3-dihydro-benzofuran-7-yl-oxy)-2-hydroxypropyl/-4-(2,4-dichlorophenyl)-piperazine, 1-/3-(2,2-dimethyl-2,3-dihydro-benzofuran-7-yl-oxy)-2-hydroxypropyl/-4-(3-chlorophenyl)-piperazine, 1-/3-(2,2-dimethyl-2,3-dihydro-benzofuran-7-yl-oxy)-2-hydroxypropyl/-4-(2-pyridyl)piperazine, 1-/3-(2,2-dimethyl-2,3-dihydro-benzofuran-7-yl-oxy)-2-hydroxypropyl/-4-(2-methoxyphenyl)-piperazine or 1-/3-(2,2-dimethyl-2,3-dihydro-benzofuran-7-yl-oxy)-2-hydroxypropyl/-4-(3-methoxyphenyl)-piperazine,
and pharmaceutically suitable acid addition salts thereof.

7. A process for the preparation of a benzofuran derivative of the formula I, wherein R¹, R², Z, X, Y, A, B and Ar are as defined in claim 1, or a pharmaceutically suitable acid addition salt thereof, characterized in that
a) a halide of the formula 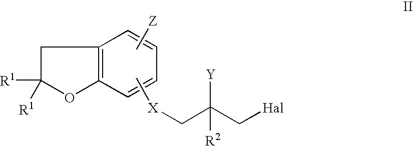
wherein R¹, R², X, Y and Z are as defined in connection with formula I, Hal represents a halo atom, is reacted with a secondary amine of the formula 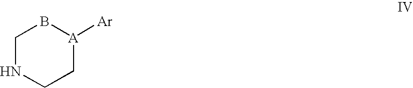
wherein A, B and Ar are as stated in connection with formula I; or
b) for the preparation of a benzofuran derivative of the formula I, wherein Y represents a hydroxy group, R¹, R², X, Z, A, B and Ar are as defined in connection with formula I, an epoxide of the formula 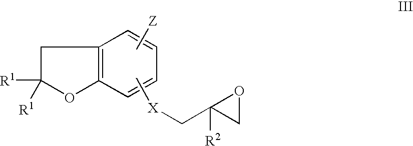
wherein R¹, R², Z and X are as defined above, is reacted with a secondary amine of the formula IV, wherein A, B and Ar are as stated above; or
c) a compound of the formula 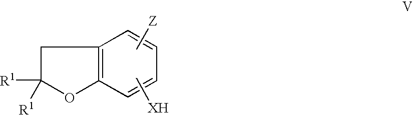
wherein R¹, X and Z are as defined in connection with formula I, is reacted with a halo compound of the formula 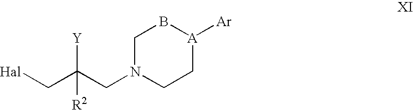
wherein R², Y, A, B and Ar are as stated in connection with formula I, Hal represents a halo atom;
d) for the preparation of a benzofuran derivative of the formula I, wherein R¹, R², X, Z, A, B and Ar are as defined in connection with formula I, a compound of the formula V, wherein R¹, X and Z are as stated above, is reacted with an epoxide of the formula 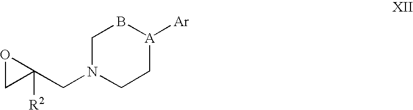
wherein R², A, B and Ar are as stated above; or
e) for the preparation of a benzofuran derivative of the formula I, wherein A forms with B a group of the formula —C═C—, R¹, R², X, Y, Z and Ar are as defined in connection with formula I, a benzofuran derivative of the formula I, wherein A stands for a group of the formula COH, B represents a methylene group, R¹, R², X, Y, Z and Ar are as stated above, is dehydrated; or
f) for the preparation of a benzofuran derivative of the formula I, wherein A represents a group of the formula COH, B stands for a methylene group, R¹, R², X, Y, Z and Ar are as defined in connection with formula I, however, Ar is other than a hydrogen atom, a ketone of the formula 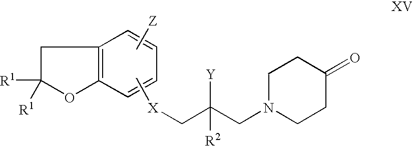
wherein R¹, R², X, Y and Z are as stated above, is reacted with an arylmagnesium halide of the formula
Hal-Mg—Ar XVI
wherein Ar is as stated above, Hal represents a halo atom, and the adduct formed is decomposed with water; or
g) for the preparation of a benzofuran derivative of the formula I, wherein A represents a group of the formula COH, B stands for a methylene group, R¹, R², X, Y, Z and Ar are as defined in connection with formula I, but Ar is other than a hydrogen atom, a ketone of the formula XV, wherein R¹ R² X, Y and Z are as stated above, is reacted with an aryl lithium compound of the formula
Li—Ar XVII
wherein Ar is as stated above, and the adduct formed is decomposed with water; or
h) for the preparation of a benzofuran derivative of the formula I, wherein A represents a group of the formula CH, B stands for a methylene group, R¹, R², X, Y, Z and Ar are as defined in connection with formula I, a compound of the formula I, wherein A forms with B a group of the formula —C═C—, R¹, R², X, Y, Z and Ar are as stated above, is hydrogenized; or
i) for the preparation of a benzofuran derivative of the formula I, wherein A represents a group of the formula CH, B stands for a methylene group, R¹, R², X, Y, Z and Ar are as defined in connection with formula I, an epoxide of the formula III, wherein R¹, R², Z and X are as stated above, is reacted with a secondary amine of the formula IV, wherein A stands for a group of the formula CHOH, B and Ar are as stated above, under dehydrating reaction conditions, and the formed compound of the formula I, wherein A forms with B a group of the formula —C═C—, R¹, R² X, Y, Z and Ar are as stated above, is hydrogenized in the reaction mixture in which it was prepared; and
if desired, an obtained base of the formula I is reacted with an inorganic or organic acid to form a pharmaceutically suitable acid addition salt thereof, or liberated from the acid addition salt with a base.

8. A pharmaceutical composition comprising a benzofuran derivative of the formula 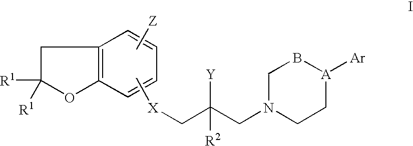
wherein
R¹ and R² represent, independently, a hydrogen atom or a C₁₋₄ alkyl group,
X stands for an oxygen atom or a sulfur atom,
Y means a hydrogen atom or a hydroxy group,
Z represents a hydrogen atom, a halo atom, a C₁₋₄ alkyl group, a C₁₋₄ alkoxy group, an amino group, a nitro group, a cyano group, a trifluoromethyl group, a group of the formula —COOR³, —NHCOR³ or —SO₂NR³R⁴, wherein
R³ stands for a hydrogen atom or a C₁₋₄ alkyl group,
R⁴ is a C₁₋₄ alkyl group, or
R³ and R⁴ form, together with the adjacent nitrogen atom, a saturated or unsaturated heterocyclic group having 5 to 10 members and optionally comprising one or more nitrogen atom(s) and/or one or more oxygen atom(s) and/or one or more sulfur atom(s) as the further heteroatom(s),
A means a group of the formula CH, COH, C—CN, C—COOR³⁴ or COR³⁴, wherein R³ and R⁴ are as defined above,
B represents a methylene group, or
A forms together with B a group of the formula —C═C—,
Ar stands for a hydrogen atom, a C₁₋₄ alkyl 1-4 group, a phenyl(C₁₋₄ alkyl) group, a biphenylyl group, a naphthyl group, wherein said latter species are optionally substituted by a C₁₋₄ alkoxy group or a C₂₋₄ alkenyl group; a partially saturated, 5- or 6-membered heterocyclic group condensed with a phenyl group and containing one or two oxygen atom(s), said heterocyclic group being optionally substituted by one to three C₁₋₄ alkyl group; a 5- or 6-membered, saturated or unsaturated hetero cyclic group containing a nitrogen atom and/or an oxygen atom and/or a sulfur atom as the heteroatom; or a phenyl group substituted by the substituents R⁵, R⁶ and R⁷, wherein
R⁵, R⁶ and R⁷ mean, independently, a hydrogen atom, a halo atom, a trifluoro-methyl group, a C₁₋₄ alkyl group, a methylenedioxy group, a phenoxy group optionally substituted by a C₁₋₄ alkoxy group or by a halo atom; a C₂₋₄ alkenyl group, a C₂₋₄ alkenyloxy group, a C₁₋₄ alkoxy group optionally substituted by a di(C₁₋₄ alkyl)amino group or by a 5- or 6-membered, saturated hetero-cyclic group containing one or two nitrogen atom(s) or a nitrogen atom and an oxygen atom, wherein said heterocyclic group is optionally substituted by a C₁₋₄ alkyl group, or
A stands for a group of the formula N—(CH₂)ₙ—Ar′, wherein
Ar′ represents a diphenylmethyl group, a pyridyl group, a pyrimidinyl group, a naphthyl group, wherein said latter group is optionally substituted by a C₁₋₄ alkoxy group or a C₂₋₄ alkenyloxy group; a partially saturated, 5- or 6-membered heterocyclic group condensed with a phenyl group and containing one or two oxygen atom(s), said hetero-cyclic group being optionally substituted by one to three C₁₋₄ alkyl group(s); or a phenyl group substituted by the substituents R⁵, R⁶ and R⁷₁ wherein R⁵, R⁶ and R⁷ are as defined above,
n has a value of 0 or 1,
or a pharmaceutically suitable acid addition salt thereof as the active ingredient and one or more conventional-carrier(s).

9. A pharmaceutical composition as claimed in claim 8, comprising a benzofuran derivative of the formula I, wherein
R¹ represents a hydrogen atom or a C₁₋₄ alkyl group,
R² stands for a hydrogen atom,
X means an oxygen atom,
Y is a hydrogen atom or a hydroxy group,
Z represents a hydrogen atom, a halo atom or a nitro group,
A stands for a group of the formula CH, COH or C—CN,
B means a methylene group, or
A forms with B a group of the formula —C═C—,
Ar represents a hydrogen atom, a benzyl group, a phenyl group substituted by substituents R⁵, R⁶ and R⁷, a biphenylyl group, a naphthyl group optionally substituted by a C₁₋₄ alkoxy group; or a thienyl group, wherein
R⁵, R⁶ and R⁷ mean, independently, a hydrogen atom, a halo atom, a trifluoro-methyl group, a C₁₋₄ alkyl group, a C₁₋₄ alkoxy group, a C₂₋₄ alkenyloxy group, a phenoxy group or a methylenedioxy group,
or a pharmaceutically suitable acid addition salt thereof as the active ingredient.

10. A pharmaceutical composition as claimed in claim 8 or 9, comprising a benzofuran derivative of the formula I, wherein
R¹ represents a methyl group,
R² stands for a hydrogen atom,
X means an oxygen atom,
Y is a hydroxy group,
Z represents a hydrogen atom,
A is a group of the formula CH, COH or C—CN,
B stands for a methylene group, or
A forms with B a group of the formula —C═C—,
Ar represents a phenyl group optionally substituted by a halo atom, a trifluoro-methyl group, a methyl group or a methoxy group; or a methoxynaphthyl group,
or a pharmaceutically suitable acid addition salt thereof as the active ingredient.

11. A pharmaceutical composition as claimed in claim 8, comprising a piperazinyl-alkylbenzofuran derivative of the formula 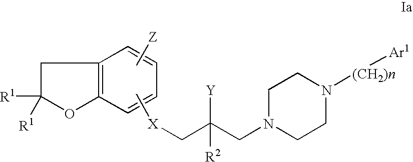
wherein
R¹ represents a C₁₋₄ alkyl group,
R² stands for a hydrogen atom,
X means an oxygen atom,
Y is a hydroxy group,
Z represents a hydrogen atom,
Ar′ represents a diphenylmethyl group, a pyridyl group, a partially saturated 5-membered heterocyclic group containing two oxygen atoms and being condensed with a phenyl group, or a phenyl group substituted by substituents R⁵, R⁶ and R⁷, wherein
R⁵, R⁶ and R⁷ mean, independently, a hydrogen atom, a halo atom, a trifluoro-methyl group, a C₁₋₄ alkyl group, a C₁₋₄ alkoxy group, or a methylenedioxy group,
n has a value of 0 or 1,
or a pharmaceutically suitable acid addition salt thereof as the active ingredient.

12. A pharmaceutical composition as claimed in claim 11, comprising a piperazinyl-alkylbenzofuran derivative of the formula Ia, wherein
R¹ represents a methyl group,
R² stands for a hydrogen atom,
X means an oxygen atom,
Y is a hydroxy group,
Z represents a hydrogen atom,
Ar′ represents a diphenylmethyl group, a pyridyl group, a benzo-1,3-dioxolanyl group or a phenyl group optionally substituted by one or two halo atom(s), one or two methyl group(s), a methylenedioxy group, a trifluoromethyl group or a methoxy group,
n has a value of 0 or 1,
or a pharmaceutically suitable acid addition salt thereof as the active ingredient.

13. A pharmaceutical composition as claimed in claim 8, comprising one of the following compounds: 1-/3-(2,2-dimethyl-2,3-dihydro-benzofuran-7-yloxy)-2-hydroxypropyl/-4-(3-trifluoro-methylphenyl)-1,2,3,6-tetrahydropyridine, 1-/3-(2,2-dimethyl-2,3-dihydro-benzofuran-7-yl-oxy)-2-hydroxypropyl/-4-hydroxy-4-(3-trifluoro-methylphenyl)piperidine, 1-/3-(2,2-dimethyl-2,3-dihydro-benzofuran-7-yl-oxy)-2-hydroxypropyl/-4-hydroxy-4-(4-fluoro-phenyl)piperidine, 1-/3-(2,2-dimethyl-2,3-dihydro-benzofuran-7-yl oxy)-2-hydroxypropyl/-4-hydroxy-4-phenyl-piperidine, 1-/3-(2,2-dimethyl-2,3-dihydro-benzofuran-7-yl oxy)-2-hydroxypropyl/-4-hydroxy-4-(3-chloro-phenyl)piperidine, 1-/3-(2,2-dimethyl-2,3-dihydro-benzofuran-7-yl-oxy)-2-hydroxypropyl/-4-hydroxy-4-(3-methoxy-phenyl)piperidine, 1-/3-(2,2-dimethyl-2,3-dihydro-benzofuran-7-yl-oxy)-2-hydroxypropyl/-4-hydroxy-4-(4-methoxy-phenyl)piperidine, 1-/3-(2,2-dimethyl-2,3-dihydro-benzofuran-7-yl-oxy)-2-hydroxypropyl/-4-(3-trifluoromethyl-phenyl)piperidine, 1-/3-(2,2-dimethyl-2,3-dihydro-benzofuran-7-yl-oxy)-2-hydroxypropyl/-4-hydroxy-4-(4-methyl-phenyl)piperidine, 1-/3-(2,2-dimethyl-2,3-dihydro-benzofuran-7-yl-oxy)-2-hydroxypropyl/-4-cyano-4-phenyl-piperidine, 1-/3-(2,2-dimethyl-2,3-dihydro-benzofuran-7-yl-oxy)-2-hydroxypropyl/-4-hydroxy-4-(4-chloro-phenyl)piperidine, 1-/3-(2,2-dimethyl-2,3-dihydro-benzofuran-7-yl-oxy)-2-hydroxypropyl/-4-hydroxy-4-(6-methoxy-naphth-2-yl)piperidine, 1-/3-(2,2-dimethyl-2,3-dihydro-benzofuran-7-yl-oxy)-2-hydroxypropyl/-4-(diphenylmethyl)-piperazine, 1-/3-(2,2-dimethyl-2,3-dihydro-benzofuran-7-yl-oxy)-2-hydroxypropyl/-4-(4-fluorophenyl)-piperazine, 1-/3-(2,2-dimethyl-2,3-dihydro-benzofuran-7-yl-oxy)-2-hydroxypropyl/-4-hydroxy-4-(3-trifluoro-methylphenyl)piperazine, 1-/3-(2,2-dimethyl-2,3-dihydro-benzofuran-7-yl-oxy)-2-hydroxypropyl/-4-(4-methoxyphenyl)-piperazine, 1-/3-(2,2-dimethyl-2,3-dihydro-benzofuran-7-yl-oxy)-2-hydroxypropyl/-4-(benzo-1,3-dioxolan-5-yl)piperazine, 1-/3-(2,2-dimethyl-2,3-dihydro-benzofuran-7-yl-oxy)-2-hydroxypropyl/-4-(4-chlorophenyl)-piperazine, 1-/3-(2,2-dimethyl-2,3-dihydro-benzofuran-7-yl-oxy)-2-hydroxypropyl/-4-benzylpiperazine, 1-/3-(2,2-dimethyl-2,3-dihydro-benzofuran-7-yl-oxy)-2-hydroxypropyl/-4-(2,4-dichlorophenyl)-piperazine, 1-/3-(2,2-dimethyl-2,3-dihydro-benzofuran-7-yl-oxy)-2-hydroxypropyl/-4-(3-chlorophenyl)-piperazine, 1-/3-(2,2-dimethyl-2,3-dihydro-benzofuran-7-yl -oxy)-2-hydroxypropyl/-4-(2-pyridyl)piperazine, 1-/3-(2,2-dimethyl-2,3-dihydro-benzofuran-7-yl-oxy)-2-hydroxypropyl/-4-(2-methoxyphenyl)-piperazine or 1-/3-(2,2-dimethyl-2,3-dihydro-benzofuran-7-yl-oxy)-2-hydroxypropyl/-4-(3-methoxyphenyl)-piperazine,
or a pharmaceutically suitable acid addition salt thereof as the active ingredient.

14. A method of treatment in which a patient suffering especially from a heart disease or a disease of the central nervous system is treated with a non-toxic dose of a benzofuran derivative of the formula 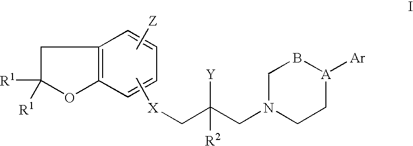
wherein
R¹ and R² represent, independently, a hydrogen atom or a C₁₋₄ alkyl group,
X stands for an oxygen atom or a sulfur atom,
Y means a hydrogen atom or a hydroxy group,
Z represents a hydrogen atom, a halo atom, a C₁₋₄ alkyl group, a C₁₋₄ alkoxy group, an amino group, a nitro group, a cyano group, a trifluoromethyl group, a group of the formula —COOR³, —NHCOR³ or —SO₂NR³R⁴, wherein
R³ stands for a hydrogen atom or a C₁₋₄ alkyl group,
R⁴ is a C₁₋₄ alkyl group, or
R³ and R⁴ form, together with the adjacent nitrogen atom, a saturated or unsaturated heterocyclic group having 5 to 10 members and optionally comprising one or more nitrogen atom(s) and/or one or more oxygen atom(s) and/or one or more sulfur atom(s) as the further heteroatom(s),
A means a group of the formula CH, COH, C—CN, C—COOR³ or COR⁴, wherein R³ and R⁴ are as defined above,
B represents a methylene group, or
A forms together with B a group of the formula —C═C—,
Ar stands for a hydrogen atom, a C₁₋₄ alkyl group, a phenyl(C₁₋₄ alkyl) group, a biphenylyl group, a naphthyl group, wherein said latter species are optionally substituted by a C₁₋₄ alkoxy group or a C₂₋₄ alkenyl group; a partially saturated, 5- or 6-membered heterocyclic group condensed with a phenyl group and containing one or two oxygen atom(s), said heterocyclic group being optionally substituted by one to three C₁₋₄ alkyl group; a 5- or 6-membered, saturated or unsaturated hetero cyclic group containing a nitrogen atom and/or an oxygen atom and/or a sulfur atom as the heteroatom; or a phenyl group substituted by the substituents R⁵, R⁶ and R⁷, wherein
R⁵R⁶ and R⁷ mean, independently, a hydrogen atom, a halo atom, a trifluoro-methyl group, a C₁₋₄ alkyl group, a methylenedioxy group, a phenoxy group optionally substituted by a C₁₋₄ alkoxy group or by a halo atom; a C₂₋₄ alkenyl group, a C₂₋₄ alkenyloxy group, a C₁₋₄ alkoxy group optionally substituted by a di(C₁₋₄ alkyl)amino group or by a 5- or 6-membered, saturated hetero-cyclic group containing one or two nitrogen atom(s) or a nitrogen atom and an oxygen atom, wherein said heterocyclic group is optionally substituted by a C₁₋₄ alkyl group, or
A stands for a group of the formula N—(CH₂)ₙAr′, wherein
Ar′ represents a diphenylmethyl group, a pyridyl group, a pyrimidinyl group, a naphthyl group, wherein said latter group is optionally substituted by a C₁₋₄ alkoxy group or a C₂₋₄ alkenyloxy group; a partially saturated, 5- or 6-membered heterocyclic group condensed with a phenyl group and containing one or two oxygen atom(s), said hetero-cyclic group being optionally substituted by one to three C₁₋₄ alkyl group(s); or a phenyl group substituted by the substituents R⁵, R⁶ and R⁷ wherein R⁵, R⁶ and R⁷ are as defined above,
n has a value of 0 or 1,
or a pharmaceutically suitable acid addition salt thereof.

15. A process for the preparation of a pharmaceutical composition having especially cardioprotective action or being suitable for the treatment of a disease of the central nervous system, characterized in that a benzofuran derivative of the formula 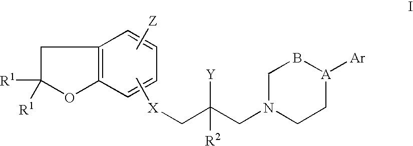
wherein
R¹ and R² represent, independently, a hydrogen atom or a C₁₋₄ alkyl group,
X stands for an oxygen atom or a sulfur atom,
Y means a hydrogen atom or a hydroxy group,
Z represents a hydrogen atom, a halo atom, a C₁₋₄ alkyl group, a C₁₋₄ alkoxy group, an amino group, a nitro group, a cyano group, a trifluoromethyl group, a group of the formula —COOR³, —NHCOR³ or —SO₂NR³R⁴, wherein
R³ stands for a hydrogen atom or a C₁₋₄ alkyl group,
R⁴ is a C₁₋₄ alkyl group, or
R³ and R⁴ form, together with the adjacent nitrogen atom, a saturated or unsaturated heterocyclic group having 5 to 10 members and optionally comprising one or more nitrogen atom(s) and/or one or more oxygen atom(s) and/or one or more sulfur atom(s) as the further heteroatom(s),
A means a group of the formula CH, COH, C—CN, C—COOR³ or COR⁴, wherein R³ and R⁴ are as defined above,
B represents a methylene group, or
A forms together with B a group of the formula —C═C—,
Ar stands for a hydrogen atom, a C₁₋₄ alkyl group, a phenyl(C₁₋₄ alkyl) group t a biphenylyl group, a naphthyl group, wherein said latter species are optionally substituted by a C₁₋₄ alkoxy group or a C₂₋₄ alkenyl group; a partially saturated, 5- or 6-membered heterocyclic group condensed with a phenyl group and containing one or two oxygen atom(s), said heterocyclic group being optionally substituted by one to three C₁₋₄ alkyl group; a 5-or 6-membered, saturated or unsaturated hetero cyclic group containing a nitrogen atom and/or an oxygen atom and/or a sulfur atom as the heteroatom; or a phenyl group substituted by the substituents R⁵, R⁶ and R⁷, wherein
R⁵, R⁶ and R⁷ mean, independently, a hydrogen atom, a halo atom, a trifluoro-methyl group, a C₁₋₄ alkyl group, a methylenedioxy group, a phenoxy group optionally substituted by a C₁₋₄ alkoxy group or by a halo atom; a C₂₋₄ alkenyl group, a C₂₋₄ alkenyloxy group, a C₁₋₄ alkoxy group optionally substituted by a di(C₁₋₄ alkyl)amino group or by a 5- or 6-membered, saturated hetero-cyclic group containing one or two nitrogen atom(s) or a nitrogen atom and an oxygen atom, wherein said heterocyclic group is optionally substituted by a C₁₋₄ alkyl group, or
A stands for a group of the formula N—(CH₂)ₙ Ar′, wherein
Ar′ represents a diphenylmethyl group, a pyridyl group, a pyrimidinyl group, a naphthyl group, wherein said latter group is optionally substituted by a C₁₋₄ alkoxy group or a C₂₋₄ alkenyloxy group; a partially saturated, 5- or 6-membered heterocyclic group condensed with a phenyl group and containing one or two oxygen atom(s), said hetero-cyclic group being optionally substituted by one to three C₁₋₄ alkyl group(s); or a phenyl group substituted by the substituents R⁵, R⁶ and R⁷ wherein R⁵, R⁶ and R⁷ are as defined above,
n has a value of 0 or 1,
or a pharmaceutically suitable acid addition salt thereof is converted to a pharmaceutical composition using one or more carrier(s) commonly employed in the manufacture of drugs.

16. A halide of the formula 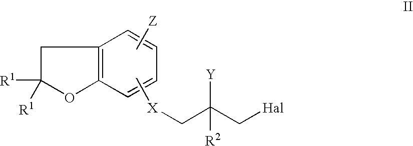
wherein
R¹ and R² represents, independently, a hydrogen atom or a C₁₋₄ alkyl group,
X stands for an oxygen atom or a sulfur atom,
Y means a hydrogen atom or a hydroxy group,
Z represents a hydrogen atom, a halo atom, a C₁₋₄ alkyl group, a C₁₋₄ alkoxy group, an amino group, a nitro group, a cyano group, a trifluoromethyl group or a group of the formula —COOR³, —NHCOR³ or —SO₂NR³R⁴ wherein
R³ stands for a hydrogen atom or a C₁₋₄ alkyl group,
R⁴ means a C₁₋₄ alkyl group, or
R³ and R⁴ form, together with the adjacent nitrogen atom, a saturated or unsaturated heterocyclic group having 5 to 10 members and optionally comprising one or more nitrogen atom(s) and/or one or more oxygen atom(s) and/or one or more sulfur atom(s),
Hal represents a halo atom.

17. A ketone of the formula 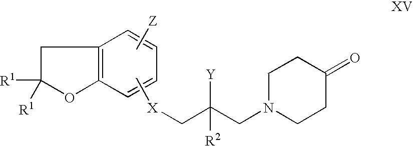
wherein
R¹ and R² represents, independently, a hydrogen atom or a C₁₋₄ alkyl group,
X stands for an oxygen atom or a sulfur atom,
Y means a hydrogen atom or a hydroxy group,
Z represents a hydrogen atom, a halo atom, a C₁₋₄ alkyl group, a C₁₋₄ alkoxy group, an amino group, a nitro group, a cyano group, a trifluoromethyl group or a group of the formula —COOR³, —NHCOR³ or —SO₂NR³R⁴. wherein
R³ stands for a hydrogen atom or a C₁₋₄ alkyl group,
R³ means a C₁₋₄ alkyl group, or
R³ and R⁴ form, together with the adjacent nitrogen atom, a saturated or unsaturated heterocyclic group having 5 to 10 members and optionally comprising one or more nitrogen atom(s) and/or one or more oxygen atom(s) and/or one or more sulfur atom(s).
